(19) United States
(12) Patent Application Publication
(10) Pub. No.: US 2019/0345489 A1
(43) Pub. Date: Nov. 14, 2019

(54) REAGENT FOR USE IN ASSESSMENT OF REMAINING VERY SMALL LESION OF NEUROBLASTOMA; AND METHOD FOR ANALYZING BIOLOGICAL SAMPLE USING SAME

(71) Applicant: National University Corporation Kobe University, Hyogo (JP)

(72) Inventor: Noriyuki Nishimura, Kobe-shi, Hyogo (JP)

(21) Appl. No.: 16/474,510

(22) PCT Filed: Dec. 20, 2017
(86) PCT No.: PCT/JP2017/045715

§ 371 Date: Jun. 27, 2019

(30) Foreign Application Priority Data
Dec. 28, 2016 (JP) 2016-255108

(51) Int. Cl. C12N 15/10; C12Q 1/686

(52) U.S. Cl. CPC C12N 15/1096; C12Q 1/686

(57) ABSTRACT

There is provided a combination of genetic markers capable of detecting minimal residual disease in neuroblastoma with high sensitivity, even in a sample with a low gene expression level. A reagent according to the present invention comprises a primer pair capable of amplifying each of genetic markers CRMP1, DBH, DDC, GAP43, ISL1, PHOX2B, and TH, by a nucleic acid amplification method, and is used for evaluating minimal residual disease in neuroblastoma. The reagent may further comprise a primer pair capable of amplifying the HPRT1 gene by the nucleic acid amplification method. A method of analyzing a biological specimen according to the present invention comprises the step of measuring an expression level of each of genetic markers CRMP1, DBH, DDC, GAP43, ISL1, PHOX2B, and TH in the biological specimen by a nucleic acid amplification method, using the above-described reagent. The nucleic acid amplification method is preferably performed by digital PCR.

### TECHNICAL FIELD

The present invention relates to a genetic marker for evaluating minimal residual disease in neuroblastoma, and use thereof. More specifically, the present invention relates to a reagent used for evaluating minimal residual disease in neuroblastoma, and a method of analyzing a biological specimen using the reagent.

### BACKGROUND ART

Neuroblastoma is a refractory childhood cancer that originates from neural crest cells, and accounts for about 10% of childhood cancers. This is the second highest incidence after brain tumor. Neuroblastoma accounts for about 15% of death causes due to childhood cancers.

Neuroblastoma is divided into low-, intermediate-, and high-risk groups, using five prognostic factors (disease stage, pathology, age, MYCN amplification, and DNA ploidy), and more than 50% of the patients are divided into the high-risk group. More than 50% of the high-risk group patients experience relapse.

Therefore, in order to improve the prognosis of neuroblastoma, particularly the prognosis of high-risk group patients, it is indispensable to accurately evaluate minimal residual disease (MRD), which is believed to be the origin of relapse. In neuroblastoma in which genes that are detected only in tumor cells have not been identified, detection of MRD has been attempted by using, as markers, several genes expressed at higher levels in tumor cells than in normal cells.

As genetic markers of MRD in neuroblastoma, TH was reported first (Non Patent Literature 1), followed by PHOX2B (Non Patent Literature 2). Furthermore, Non Patent Literature 3 reported five genetic markers, CHGA, DCX, DDC, PHOX2B, and TH. Non Patent Literature 4 reported four genetic markers, B4GALNT (GD2 synthase), CCND1, ISL1, and PHOX2B. Non Patent Literature 5 reported five genetic markers, CHRNA3, DDC, GAP43, PHOX2B, and TH.

On the other hand, Non Patent Literature 6 proposed a MRD detection protocol that investigates spheres of neuroblastoma cells suspension-cultured to concentrate cancer stem cells constituting MRD in vivo, rather than adherent-cultured parental neuroblastoma cells conventionally used in genetic marker search, and scores the cases as MRD-positive where the expression of any of the 11 genetic markers CHRNA3, CRMP1, DBH, DCX, DDC, GABRB3, GAP43, ISL1, KIF1A, PHOX2B, and TH exceeds the normal range. Non Patent Literature 7 reported that two cases were evaluated as MRD-positive using the 11 genetic markers, at a stage earlier than the clinical diagnosis of relapse or regrowth. In Non Patent Literatures 6 and 7 described above, real time PCR is used to measure the genetic markers.

### CITATION LIST

### Non Patent Literature

### Non Patent Literature 1: International Journal of Cancer (1994) 57: 671-675.

### Non Patent Literature 2: Journal of Clinical Oncology (2008) 26: 5443-5449.

### Non Patent Literature 3: Lancet Oncology (2013) 14: 999-1008.

### Non Patent Literature 4: Journal of Clinical Oncology (2015) 33: 755-763.

### Non Patent Literature 5: European Journal of Cancer (2016) 54: 149-158.

### Non Patent Literature 6: Oncology Reports (2013) 29: 1629-1636.

### Non Patent Literature 7: Oncology Letters (2016) 12: 1119-1123.

### SUMMARY OF INVENTION

### Technical Problem

As described above, various markers have been reported as MRD markers of neuroblastoma. Regarding TH reported first in Non Patent Literature 1 as a MRD marker of neuroblastoma, many cases have been reported which became TH-negative when TH was used alone, and in which MRD could not be detected; subsequently, even regarding PHOX2B reported in Non Patent Literature 2 as a MRD marker with fewer negative cases, some negative cases were found when PHOX2B was used alone. This suggests that neuroblastoma, among tumors, has a characteristic problem in that its heterogeneity is particularly remarkable. To overcome the heterogeneity characteristic of neuroblastoma, detection of MRD using a plurality of MRD markers has been attempted. In the MRD markers of neuroblastoma reported in each of Non-Patent Literatures 3 to 5, markers different from previous markers were combined, and it is unclear to what extent false negatives occur; however, because these markers were selected based on the expression of conventional adherent-cultured neuroblastoma cell lines, they are considered to have room for improvement in terms of specificity.

The MRD markers of neuroblastoma reported in Non-Patent Literatures 6 and 7 were selected from markers with high expression levels in spheres of neuroblastoma cells, and are preferable because of their high specificity. Furthermore, to overcome the heterogeneity characteristic of neuroblastoma, it is common knowledge in the art that the screening sensitivity needs to be increased by increasing the number of combined markers. The combination of as many as 11 markers with high expression levels in spheres of neuroblastoma cells is also preferable in terms of reducing false negatives. However, the use of as many as 11 markers as analytes leads to poor test efficiency, and becomes a very high barrier to clinical application.

As described above, in MRD detection in neuroblastoma, the remarkable heterogeneity characteristic of neuroblastoma has made it particularly difficult to achieve a reduction in false negatives and clinical applicability simultaneously. Accordingly, it is an object of the present invention to provide a combination of a small number of MRD markers of neuroblastoma that is suitable for clinical application, while reducing false negatives.

### Solution to Problem

As a result of extensive research, the present inventor has found specific seven genetic markers with a particularly high ability to detect MRD in neuroblastoma. The present invention has been completed as a result of further research based on this finding.

The present invention includes a reagent for evaluating minimal residual disease in neuroblastoma and a method of analyzing a biological specimen using the reagent.

(1)

A reagent according to the present invention comprises a primer pair capable of amplifying each of genetic markers CRMP1, DBH, DDC, GAP43, ISL1, PHOX2B, and TH, by a nucleic acid amplification method, and is used for evaluating minimal residual disease in neuroblastoma.

The above-described genetic markers denoted as CRMP1, DBH, DDC, GAP43, ISL1, PHOX2B, and TH, respectively, include polynucleotides consisting of nucleotide sequences as set forth in SEQ ID NOS: 1 to 7 shown below, and polynucleotides consisting of nucleotide sequences having at least 70% homology with the polynucleotides, and functionally equivalent thereto.

While the genetic markers according to the present invention are a combination of only seven genetic markers, each genetic marker has a particularly high ability to detect minimal residual disease in neuroblastoma, thereby reducing false negatives, as well as facilitating clinical application. Furthermore, because the annealing temperatures of all the primer pairs corresponding to the respective genes can be readily equalized, any of the seven genetic markers can be amplified equally efficiently. This also facilitates clinical application of the genetic marker set according to the present invention.

(2)

The reagent according to (1) above may further comprise a primer pair capable of amplifying at least any of HPRT1, HMBS, GUSB, TBP, and B2M as reference genes, by the nucleic acid amplification method.

In (2) above as well as (3) and (4) below, the reference genes denoted as HPRT1, HMBS, GUSB, TBP, and B2M, respectively, include polynucleotides consisting of nucleotide sequences as set forth in SEQ ID NOS: 8 to 12 shown below, and polynucleotides consisting of nucleotide sequences having at least 70% homology with the polynucleotides, and functionally equivalent thereto.

Through the combined use of the primer pairs for these reference genes, the occurrence of minimal residual disease in neuroblastoma can be more accurately evaluated.

(3)

The reagent according to (1) above may further comprise a primer pair capable of amplifying at least any of HPRT1, HMBS, GUSB, and TBP as reference genes, by the nucleic acid amplification method.

Because these specific reference genes are genes with low expression levels, minimal residual disease in neuroblastoma can be detected with improved sensitivity, even in a sample with a low gene expression level. Therefore, through the combined use of the primer pairs for these specific reference genes, the occurrence of minimal residual disease in neuroblastoma can be more satisfactorily evaluated, even if the level of genetic marker expression is low.

(4)

The reagent according to (1) above may further comprise a primer pair capable of amplifying HPRT1 as a reference gene, by the nucleic acid amplification method.

Because the reference gene HPRT1 is a gene with a low expression level, minimal residual disease in neuroblastoma can be detected with improved sensitivity, even in a sample with a low gene expression level. Additionally, the reference gene HPRT1, in particular, shows low variations in expression level between bone marrow samples and peripheral blood samples. Thus, whether in a bone marrow sample or a peripheral blood sample, and even in a sample with a low gene expression level, minimal residual disease in neuroblastoma can be accurately detected.

(5)

The reagent according to (1) to (4) above may further comprise a probe capable of hybridizing with any of the genetic markers under stringent conditions.

In this manner, the genetic marker can be detected by hybridization with the probe.

(6)

The reagent according to (5) above may further comprise, when the reagent further comprises a primer pair capable of amplifying a reference gene by the nucleic acid amplification method, a probe capable of hybridizing with the reference gene under stringent conditions.

In this manner, the reference gene, together with the genetic marker, can be detected by hybridization with the probe.

(7)

A method of analyzing a biological specimen according to the present invention comprises a measurement step of measuring an expression level of each of genetic markers CRMP1, DBH, DDC, GAP43, ISL1, PHOX2B, and TH in the biological specimen by a nucleic acid amplification method, using the reagent according to any one of (1) to (6) above. The expression level of the genetic marker is correlated with a level of occurrence of minimal residual disease in neuroblastoma.

While the genetic markers according to the present invention are a combination of only seven genetic markers, each genetic marker has a particularly high ability to detect minimal residual disease in neuroblastoma, thereby reducing false negatives, as well as facilitating clinical application. Furthermore, because the annealing temperatures of all the primer pairs corresponding to the respective genes can be readily equalized, any of the seven genetic markers can be amplified equally efficiently. This also facilitates clinical application of the genetic marker set according to the present invention.

(8)

The method of analyzing a biological specimen according to (7) above may further comprise an evaluation step of evaluating whether or not the expression level of at least one of the genetic markers is not lower than a threshold level.

In the present invention, because any of the seven genetic markers is amplified equally efficiently, minimal residual disease in neuroblastoma can be determined as positive when the expression level of at least one of the seven genetic markers is not lower than the threshold level, as described above.

(9)

In the method of analyzing a biological specimen according to (8) above, in the evaluation step, when the expression level of at least one of the genetic markers is not lower than the threshold level, minimal residual disease in neuroblastoma in the biological specimen may be determined as positive.

In this manner, minimal residual disease in neuroblastoma in a patient from which the biological specimen is derived can be accurately diagnosed.

(10)

In the method of analyzing a biological specimen according to any one of (7) to (9) above, the nucleic acid amplification method in the measurement step may be digital PCR.

In this case, minimal residual disease in neuroblastoma can be detected with high sensitivity, even in a sample with a low gene expression level. Furthermore, the variation in measurement results can be reduced even between different analytical subjects, analytical periods, analytical devices, protocols (such as reaction times or reaction temperatures), and the like, such that minimal residual disease in neuroblastoma can be accurately detected.

### Advantageous Effects of Invention

According to the present invention, there is provided a combination of high-sensitivity MRD markers of neuroblastoma that is suitable for clinical application while reducing false negatives, such that minimal residual disease in neuroblastoma can be accurately evaluated in more patients.

### BRIEF DESCRIPTION OF DRAWINGS

FIG. 1 shows the number of positive samples out of 252 samples evaluated for MRD using the seven markers according to the present invention (CRMP1, DBH, DDC, GAP43, ISL1, PHOX2B, and TH) and other four markers (DCX, CHRNA3, KIF1A, and GABRB3).

FIG. 2 shows the number of samples, out of the 252 samples in FIG. 1, that became single marker-positive with respect to the seven markers and the other four markers.

### DESCRIPTION OF EMBODIMENTS

[1. Reagent for Evaluating Minimal Residual Disease in Neuroblastoma]

The reagent according to the present invention is used for evaluating minimal residual disease in neuroblastoma, and includes polynucleotide molecules used as primer pairs capable of amplifying the seven genetic markers described below by the nucleic acid amplification method. In addition to the polynucleotide molecules used as primer pairs, the reagent may further include polynucleotide molecules used as probes capable of hybridizing with the genetic markers under stringent conditions.

As used herein, the term “polynucleotide molecule” is used to include DNA, RNA, and PNA (peptide nucleic acid). In a preferred embodiment of the present invention, the polynucleotide molecule is DNA or RNA.

[1-1. Genetic Markers]

The genetic marker according to the present invention includes the following seven genetic markers: the gene denoted as CRMP1; the gene denoted as DBH; the gene denoted as DDC; the gene denoted as GAP43; the gene denoted as ISL1; the gene denoted as PHOX2B; and the gene denoted as TH (hereinafter, each gene is simply denoted as CRMP1, DBH, DDC, GAP43, ISL1, PHOX2B, or TH, respectively). The expression level of each of these genetic markers is increased when minimal residual disease in neuroblastoma is present.

The combination of these seven genetic markers has high sensitivity for detecting minimal residual disease in neuroblastoma by nucleic acid amplification, and has a reduced number of false negatives of minimal residual disease in neuroblastoma. These genetic markers are thus useful as indices of minimal residual disease in neuroblastoma. Therefore, minimal residual disease in neuroblastoma can be detected with high sensitivity by measuring the expression levels of these seven genetic markers by the nucleic acid amplification method.

These seven genetic markers, even when each is used as a single marker, have the capability of detecting minimal residual disease in neuroblastoma, and even though only seven genetic markers are combined, the combination has a screening sensitivity that overcomes the remarkable heterogeneity characteristic of neuroblastoma. Because the number of genetic markers is seven, even if the number of reference genes is included, the genes can be amplified simultaneously in many nucleic acid amplification apparatuses, which facilitates clinical application.

The combination of the seven genetic markers CRMP1, DBH, DDC, GAP43, ISL1, PHOX2B, and TH is useful whether the sample to be analyzed is a peripheral blood sample or a bone marrow sample. Among these genetic markers, PHOX2B, in particular, tends to have high detection sensitivity in a bone marrow sample, and CRMP1, in particular, tends to have high detection sensitivity in a peripheral blood sample.

Details of the sequence information of each of the genetic markers according to the present invention can be acquired from known databases. Examples of the sequences include the following:
as CRMP1 (collapsin response mediator protein 1), the sequence corresponding to Accession Number NM_001014809 in the RefSeq database of the U.S. National Center for Biotechnology Information (NCBI);
as DBH (dopamine β-hydroxylase), the sequence corresponding to Accession Number NM_000787 in the same database;
as DDC (dopa decarboxylase), the sequence corresponding to Accession Number NM_000790 in the same database;
as GAP43 (growth-associated protein 43), the sequence corresponding to Accession Number NM_001130064 in the same database;
as ISL1 (ISL LIM homeobox 1), the sequence corresponding to Accession Number NM_002202 in the same database;
as PHOX2B (paired-like homeobox 2b), the sequence corresponding to Accession Number NM_003924 in the same database; and
as TH (tyrosine hydroxylase), the sequence corresponding to Accession Number NM_199292 in the same database.

Specific nucleotide sequences encoding the genetic markers according to the present invention include the following sequences:
the nucleotide sequence of CRMP1 may be as set forth in SEQ ID NO: 1;
the nucleotide sequence of DBH may be as set forth in SEQ ID NO: 2;
the nucleotide sequence of DDC may be as set forth in SEQ ID NO: 3;
the nucleotide sequence of GAP43 may be as set forth in SEQ ID NO: 4;
the nucleotide sequence of ISL1 may be as set forth in SEQ ID NO: 5;
the nucleotide sequence of PHOX2B may be as set forth in SEQ ID NO: 6; and
the nucleotide sequence of TH may be as set forth in SEQ ID NO: 7.

The nucleotide sequences encoding the genetic markers according to the present invention may be nucleotide sequences having homology with the above-described sequences, as long as they serve as indices of minimal residual disease in neuroblastoma. Preferably, the genetic markers according to the present invention may include polynucleotides having at least 70% homology with the nucleotide sequences as set forth in SEQ ID NOS: 1 to 7 described above, and functionally equivalent thereto. The sequence homology of these polynucleotides is more preferably 75% or higher, even more preferably 80% or higher, still more preferably 85% or higher, even more preferably 90% or higher, and still more preferably 95% or higher.

The phrase “functionally equivalent” means that when minimal residual disease in neuroblastoma is present, the expression levels of the polynucleotides are increased equivalently to those of the specific polynucleotides having the nucleotide sequences as set forth in SEQ ID NOS: 1 to 7 mentioned above. The functional equivalency can be readily determined by, for example, measuring the expression levels of the polynucleotides by using the below-described probes or primers, determining the correlations between the expression levels and minimal residual disease in neuroblastoma by a known statistical technique, and comparing the results with those of the above-described specific polynucleotides.

The homology of the above-described nucleotide sequences can be determined by a known technique (the same applies below). Specific examples of such techniques include the algorithm BLAST [Proc. Natl. Acad. Sci. USA, 90, 5873-5877 (1993)] by Karlin and Altschul. The program called BLASTN or BLASTX has also been developed based on this algorithm [Altschul et al. J. Mol. Biol., 215, 403-410 (1990)], and this program can also be used in the present invention. Other preferable techniques include the technique using the genetic information processing software GENETYX (manufactured by Genetyx Corporation). When GENETYX is used, analysis using BLAST as well as homology analysis using the Lipman-Pearson method can be performed; thus, GENETYX can be advantageously used for homology determination in the present invention.

The above-described seven genetic markers have high sensitivity for detecting minimal residual disease in neuroblastoma, and the expression level of each of the genetic markers is increased when the level of minimal residual disease in neuroblastoma is increased. That is, the expression level and the level of occurrence of minimal residual disease in neuroblastoma are correlated with each other. Therefore, minimal residual disease in neuroblastoma can be evaluated by measuring the expression levels of all these seven genetic markers by using the below-described polynucleotide molecules, and evaluating minimal residual disease in neuroblastoma based on the measured expression levels.

[1-2. Reference Genes]

Examples of the reference gene include the gene denoted as HPRT1, the gene denoted as HMBS, the gene denoted as GUSB, the gene denoted as TBP, and the gene denoted as B2M (hereinafter, each gene is simply denoted as HPRT1, HMBS, GUSB, TBP, or B2M, respectively). At least one reference gene can be selected from these genes.

Among the above, the reference gene is preferably selected from the group consisting of the HPRT1 gene, the HMBS gene, the GUSB gene, and the TBP gene in that these genes show appropriately low expression levels, and thus, can detect MRD with improved sensitivity even in a sample with a low level of genetic marker expression. Furthermore, among these reference genes, the reference gene is most preferably the HPRT1 gene in that the gene shows low variations in expression level between bone marrow samples and peripheral blood samples, and thus, is highly useful for both a bone marrow-derived biological specimen and a peripheral blood-derived biological specimen.

Details of the sequence information of the reference genes can be acquired from known databases. Examples of the sequences include the following:
as HPRT1 (hypoxanthine phosphoribosyltransferase 1), the sequence corresponding to Accession Number NM_000194 in the RefSeq database of the U.S. National Center for Biotechnology Information (NCBI);
as HMBS (hydroxymethylbilane synthase), the sequence corresponding to Accession Number NM_000190 in the same database;
as GUSB (glucuronidase beta), the sequence corresponding to Accession Number NM_000181 in the same database;
as TBP (TATA-binding protein), the sequence corresponding to Accession Number NM_003194 in the same database; and
as B2M (beta-2-microglobulin), the sequence corresponding to Accession Number NM_004048 in the same database.

Specific nucleotide sequences encoding the reference genes include the following sequences:
the nucleotide sequence of HPRT1 may be as set forth in SEQ ID NO: 8;
the nucleotide sequence of HMBS may be as set forth in SEQ ID NO: 9;
the nucleotide sequence of GUSB may be as set forth in SEQ ID NO: 10;
the nucleotide sequence of TBP may be as set forth in SEQ ID NO: 11; and
the nucleotide sequence of B2M may be as set forth in SEQ ID NO: 12.

The nucleotide sequences encoding the reference genes may be nucleotide sequences having homology with the above-described sequences, as long as they serve as endogenous controls. Preferably, the reference genes may include polynucleotides having at least 70% homology with the above-described nucleotide sequences, and functionally equivalent to the above-described respective genes. The sequence homology of these polynucleotides is more preferably 75% or higher, even more preferably 80% or higher, still more preferably 85% or higher, even more preferably 90% or higher, and still more preferably 95% or higher.

The phrase “functionally equivalent” means that in the case of HPRT1, HMBS, GUSB, TBP, and B2M, the polynucleotides show low variations in measurement values among samples equivalently to those of the specific polynucleotides having the nucleotide sequences as set forth in SEQ ID NOS: 8 to 12 mentioned above; in the case of HPRT1, HMBS, GUSB, and TBP, the polynucleotides further show appropriately low expression levels; and in the case of HPRT1, the polynucleotide further shows low variations in measurement values between bone marrow samples and peripheral blood samples. The functional equivalency can be readily determined by, for example, measuring the expression levels of the polynucleotides by using the below-described probes or primers, determining the expression levels, and the correlations between the expression levels and minimal residual disease in neuroblastoma by a known statistical technique, and comparing the results with those of the above-described specific polynucleotides.

[1-3. Primer Pairs]

The primer pair according to the present invention is a pair of polynucleotide molecules capable of amplifying the nucleotide sequence encoding each of the above-described genetic markers. The reagent according to the present invention includes a total of seven primer pairs for the respective seven genetic markers CRMP1, DBH, DDC, GAP43, ISL1, PHOX2B, and TH.

Each of these primer pairs contains a sense primer and an anti-sense primer each designed to amplify the nucleotide sequence encoding the above-described respective genetic marker. The anti-sense primer is a polynucleotide molecule that hybridizes under stringent conditions to the polynucleotide to be amplified, and the sense primer is a polynucleotide molecule that hybridizes under stringent conditions to the complementary strand of the polynucleotide to be amplified. Specific designing of the primer pair can be appropriately performed by a person skilled in the art, based on the nucleotide sequence of the region to be amplified. For example, the primer pair may be designed such that one of the primers has the sequence of the 5′ end portion in the nucleotide sequence of the region to be amplified, and the other primer has the sequence of the 5′ end portion in the nucleotide sequence of the complementary strand of the region to be amplified.

In the present invention, the selection of the above-described seven genetic markers allows the annealing temperatures of all the primer pairs corresponding to the respective seven genetic markers to be equalized satisfactorily. Thus, when the seven genetic markers are measured simultaneously with the same heat source, the amplification efficiency for the regions to be amplified can be equalized satisfactorily. Because any of the seven genetic markers is efficiently amplified, the minimal residual disease measurement sensitivity can be improved even in a sample with a low gene expression level. This also facilitates clinical application of the genetic marker set according to the present invention.

The length and specific sequences of the primers are not particularly limited, and can be appropriately determined by a person skilled in the art. For example, for simultaneous measurement of more than one, preferably all, of the seven genetic markers, the length and specific sequences of the primers may be designed to achieve a Tm value at which the annealing temperature conditions are equalized.

The primers may be, for example, 18 or more and 30 or less nucleotides in length, and preferably 18 or more and 26 or less nucleotides in length.

Specific sequences of the primers include the following:

primers for CRMP1
(SEQ ID NO: 13)
5′-ccaatccctttatgctgacg-3′ (sense)
(SEQ ID NO: 14)
5′-ggaacgattaagttctctcctatttg-3′ (anti-sense)
primers for DBH
(SEQ ID NO: 15)
5′-tggggacactgcctattttg-3′ (sense)
(SEQ ID NO: 16)
5′-ttctggggtcctctgcac-3′ (anti-sense)
primers for DDC
(SEQ ID NO: 17)
5′-ctggagaagggggaggagt-3′ (sense)
(SEQ ID NO: 18)
5′-gccgatggatcactttggt-3′ (anti-sense)
primers for GAP43
(SEQ ID NO: 19)
5′-gaggatgctgctgccaag-3′ (sense)
(SEQ ID NO: 20)
5′-ggcactttccttaggtttggt-3′ (anti-sense)
primers for ISL1
(SEQ ID NO: 21)
5′-aaggacaagaagcgaagcat-3′ (sense)
(SEQ ID NO: 22)
5′-ttcctgtcatcccctggata-3′ (anti-sense)
primers for PHOX2B
(SEQ ID NO: 23)
5′-ctaccccgacatctacactcg-3′ (sense)
(SEQ ID NO: 24)
5′-ctcctgcttgcgaaacttg-3′ (anti-sense)
primers for TH
(SEQ ID NO: 25)
5′-tcagtgacgccaaggaca-3′ (sense)
(SEQ ID NO: 26)
5′-gtacgggtcgaacttcacg-3′ (anti-sense)

The reagent according to the present invention may include, in addition to the primer pairs capable of amplifying the above-described seven genetic markers, a primer pair capable of amplifying any of the above-described reference genes.

The length and specific sequences of the primers capable of amplifying the reference gene are not also particularly limited, and can be appropriately determined by a person skilled in the art. For example, for simultaneous measurement of more than one, preferably all, of the seven genetic markers, the length and specific sequences of the primers may be designed to achieve a Tm value at which the annealing temperature conditions are equalized to those of the above-described seven genetic markers.

The primers may be, for example, 18 or more and 30 or less nucleotides in length, and preferably 18 or more and 26 or less nucleotides in length.

Specific sequences of the primers for the reference genes include the following:

primers for HPRT1
(SEQ ID NO: 27)
5′-tgaccttgatttattttgcatacc-3′ (sense)
(SEQ ID NO: 28)
5′-cgagcaagacgttcagtcct-3′ (anti-sense)
primers for HMBS
(SEQ ID NO: 29)
5′-ctgaaagggccttcctgag-3′ (sense)
(SEQ ID NO: 30)
5′-cagactcctccagtcaggtaca-3′ (anti-sense)
primers for GUSB
(SEQ ID NO: 31)
5′-cgccctgcctatctgtattc-3′ (sense)
(SEQ ID NO: 32)
5′-tccccacagggagtgtgtag-3′ (anti-sense)
primers for TBP
(SEQ ID NO: 33)
5′-gaacatcatggatcagaacaaca-3′ (sense)
(SEQ ID NO: 34)
5′-atagggattccgggagtcat-3′ (anti-sense)
primers for B2M
(SEQ ID NO: 35)
5′-ttctggcctggaggctatc-3′ (sense)
(SEQ ID NO: 36)
5′-tcaggaaatttgactttccattc-3′ (anti-sense)

The primers may further include an additional sequence suitable for detection of the region to be amplified (specifically, a sequence not complementary to genomic DNA), for example, a linker sequence.

The primers may be labeled with a suitable labeling agent, for example, a radioactive isotope (such as ¹²⁵I, ¹³¹I, ³H, or ¹⁴C), an enzyme (such as β-galactosidase, β-glucosidase, alkaline phosphatase, peroxidase, or malate dehydrogenase), a fluorescent substance (such as fluorescamine, fluorescein isothiocyanate, Cy3, or Cy5), or a luminescent substance (such as luminol, a luminol derivative, luciferin, or lucigenin).

Furthermore, the primers, each separately, or in a mixed state as long as the function of each primer is not impaired, may be dissolved in water or a suitable buffer (such as TE buffer or Tris-HCl buffer) at a suitable concentration (for example, 1 μM or more and 50 μM or less at a concentration of 2× or more and 20× or less), and can be stored at about −20° C.

[1-4. Probes]

The probe that may be included in the reagent according to the present invention is a polynucleotide molecule capable of detecting the sequence encoding any of the above-described genetic markers or the sequence of the complementary strand thereof, for detecting minimal residual disease in neuroblastoma using the genetic markers according to the present invention as indices. Specifically, a polynucleotide molecule can be used that is capable of hybridizing under stringent conditions with the polynucleotide constituting any of the above-described genetic markers or the polynucleotide of the complementary strand thereof.

The reagent according to the present invention may further include a polynucleotide molecule capable of detecting the sequence encoding any of the reference genes or the sequence of the complementary strand thereof. Specifically, the reagent may include, as a probe, a polynucleotide molecule capable of hybridizing under stringent conditions with the polynucleotide constituting any of the above-described reference genes or the polynucleotide of the complementary strand thereof.

The term “complementary” means that two nucleotides can be paired with each other under hybridization conditions, for example, the relationship between adenine (A) and thymine (T) or uracil (U), or the relationship between cytosine (C) and guanine (G).

The term “hybridizing” means that the polynucleotide molecule hybridizes to the genetic marker or reference gene under general hybridization conditions (i.e., under annealing conditions in general PCR), preferably stringent hybridization conditions, and does not hybridize to a polynucleotide molecule other than the genetic marker or reference gene.

The term “stringent conditions” includes, for example, the conditions described in Current Protocols in Molecular Biology, John Wiley & Sons, 6.3.1-6.3.6, 1999, such as hybridization in 6×SSC (sodium chloride/sodium citrate)/45° C., followed by washing in 0.2×SSC/0.1% SDS/50-65° C. once or more; a person skilled in the art can appropriately select hybridization conditions that provide stringency equivalent thereto.

Furthermore, the polynucleotide molecule that hybridizes with the genetic marker or reference gene according to the present invention needs not be fully complementary to the genetic marker or reference gene, as long as it is capable of gene-specific hybridization. Preferably, however, the polynucleotide molecule is constructed to include all or a portion of the sequence of the polynucleotide molecule complementary to the genetic marker or reference gene.

The probe according to the present invention may be prepared as a synthetic oligonucleotide using a commercially available oligonucleotide synthesizer, for example, or as a double-stranded DNA fragment obtained by restriction enzyme treatment, for example.

The probe according to the present invention may be, for example, 18 or more and 30 or less nucleotides in length, and preferably 18 or more and 26 or less nucleotides in length.

The probe according to the present invention may be labeled with a suitable labeling agent, for example, a radioactive isotope (such as ¹²⁵I, ¹³¹I, ³H, or ¹⁴C), an enzyme (such as β-galactosidase, β-glucosidase, alkaline phosphatase, peroxidase, or malate dehydrogenase), a fluorescent substance (such as fluorescamine, fluorescein isothiocyanate, Cy3, or Cy5), or a luminescent substance (such as luminol, a luminol derivative, luciferin, or lucigenin).

Alternatively, in the probe according to the present invention, a quencher (such as MGB or TAMRA) that absorbs fluorescence energy emitted by a fluorescent substance (such as FAM or VIC) may be further attached in the proximity of the fluorescent substance. More specifically, the probe according to the present invention may be constructed as a probe (TaqMan probe) in which the fluorescent substance is attached to the 5′ end, the quencher is attached to the 3′ end, and the 3′ end is phosphorylated. In this case, in the detection reaction, the fluorescent substance and the quencher are separated, whereby fluorescence is detected.

The reagent according to the present invention may further include other components necessary for nucleic acid amplification, in addition to the above-described components mentioned as primers, or components mentioned as primers and probes. Examples of the other components include one or more components selected from, for example, nucleic acid synthetases, nucleic acid synthesis substrates, buffers, and labels (for example, when the primers and/or probes are contained in an unlabeled manner). The reagent according to the present invention may be provided as a kit item in which the components are individually packaged, or may be provided as a kit item in which any of a plurality of the components are mixed.

[2. Method of Analyzing Biological Specimen for Evaluating Minimal Residual Disease in Neuroblastoma]

As described above, the seven genetic markers according to the present invention are useful as indices of minimal residual disease in neuroblastoma. Therefore, the method of analyzing a biological specimen according to the present invention includes a measurement step of measuring an expression level of each of these seven genetic markers in the biological specimen by a nucleic acid amplification method. The expression level of the genetic marker is correlated (positively correlated) with a level of occurrence of minimal residual disease in neuroblastoma.

[2-1. Biological Specimen]

The biological specimen may be a sample derived from a neuroblastoma patient. The neuroblastoma patient may be a patient in any stage of neuroblastoma. Specific stages include at the time of initial diagnosis, after the completion of remission induction therapy, after surgery, after the completion of continuous therapy including stem cell transplantation, after radiation treatment, at the time of follow-up after maintenance therapy, and at the time of relapse diagnosis. The present invention is particularly useful when the subject is a high-risk group patient.

The biological specimen is not particularly limited as long as it is a specimen containing RNA from the subject, and can be appropriately selected according to the type of the detection method to be used. For example, the biological specimen may be biological tissue collected from the subject, and specifically, may be a nucleic acid specimen such as total RNA or mRNA prepared from bone marrow aspirate or peripheral blood in accordance with a conventional method.

[2-2. Measurement Step (Nucleic Acid Amplification)]

In the measurement step, using the nucleic acid specimen as a template, nucleic acid amplification is performed with the above-described primer pairs, and the expression levels of the resulting amplicons are measured.

As the nucleic acid amplification method, any method known in the art may be used. For example, a general PCR method, the real time PCR method, or the digital PCR method can be used as the nucleic acid amplification method. Preferably, the digital PCR method can be used as the nucleic acid amplification method. Preferred achievement of accurate evaluation of minimal residual disease in neuroblastoma is done by detecting a very low level of genetic marker expression. The use of digital PCR is particularly suitable for detecting minimal residual disease in neuroblastoma in that it can detect with high sensitivity even a sample with a low level of genetic marker expression. Additionally, digital PCR is also preferable in that the variation in measurement results is low even between different analytical subjects, analytical periods, analytical devices, protocols (such as reaction times or reaction temperatures), and the like, such that minimal residual disease in neuroblastoma can be accurately detected. The use of digital PCR, therefore, is also preferable in terms of high versatility and high reliability in evaluating minimal residual disease in neuroblastoma.

In digital PCR, initially, a large volume of a starting specimen is partitioned into smaller sub-volumes of specimens (partitioned specimens). The partitioned specimens are prepared to contain on average a single copy of a target. In this manner, the number of polynucleotide molecules present in a partitioned specimen becomes zero (negative) or one (positive), whereby digitality is achieved. By counting the number of positives in the partitioned specimens, the starting copy number of the target in the starting specimen can be estimated. That is, the genetic marker to be amplified can be quantified. Therefore, the target can be quantified even if the concentration of the target in the biological specimen is low.

In order to adjust the partitioned specimens to the proper concentration for achieving digitality, a multiple serial dilution method may be used for the starting specimen, and the volumes of the partitioned specimens may be determined by any PCR apparatus.

As digital PCR, droplet-based digital droplet PCR (ddPCR) is preferably used. The ddPCR method specifically includes a digital dilution step or droplet generation step, a PCR amplification step, a detection step, and an analysis step. In the droplet generation step, a plurality of droplets each containing reagents necessary for nucleic acid amplification are generated. In the PCR amplification step, the droplets (or large reaction volume specimens containing the droplets) are subjected to thermal cycling conditions appropriate for amplification of the target. In the detection step, the droplets (or large reaction volume specimens containing the droplets) are identified as those containing the PCR product or those not containing the PCR product. In the analysis step, the target concentration, absolute quantity (absolute quantity of the genetic marker), or relative quantity (of the genetic marker relative to the reference gene) is deduced.

Furthermore, in the present invention, the step of hybridizing the above-described probe to the nucleic acid in the biological specimen may be included. For example, when the detection of amplicons is performed based on labeling, the above-described labeled probe may be used. As one example, in the case where a TaqMan probe is used, the TaqMan probe hybridizes to a genetic marker, and when the elongation reaction from a primer reaches the hybridization region, the fluorescent labeling substance is released by the action of the Taq DNA polymerase. The released fluorescent labeling substance emits fluorescence by being released from the quenching action of the quencher.

[2-3. Evaluation Step]

As described above, in the present invention, because the annealing temperatures of all the primer pairs corresponding to the respective seven genetic markers can be readily equalized satisfactorily, any of the seven genetic markers can be efficiently amplified. Thus, in the present invention, an evaluation step can be performed in which it is evaluated whether or not the expression level of at least one genetic marker of the measured seven genetic markers is not lower than a preset threshold level, with reference to the expression levels of the genetic markers in a control not having minimal residual disease in neuroblastoma. When the expression level of the at least one genetic marker is not lower than the threshold level, minimal residual disease in neuroblastoma can be determined as positive for the biological specimen. Therefore, when the expression level of the at least one genetic marker is not lower than the threshold level, minimal residual disease in neuroblastoma can be determined as positive for a patient from which the biological specimen is derived.

The threshold level of the expression level of the genetic marker can be set by a known statistical technique, with reference to a quantitative value of the genetic marker measured in advance by the above-described technique. Examples of specific techniques for setting the threshold level include a method in which the threshold level is deduced as {the average value of the expression level of the genetic marker in control cells±n×standard deviation}; and the ROC (Receiver Operating Characteristic) analysis method. Specifically, reference may be made to the threshold levels that are set in Journal of Clinical Oncology 2008; 26: 5443-5449.

In accordance with the present invention, minimal residual disease in neuroblastoma can be detected by using the combination of the seven MRD markers of neuroblastoma having high sensitivity, which is suitable for clinical application while reducing false negatives. Because the seven markers have been selected such that the annealing temperatures of all the primer pairs corresponding to the respective genes are equalized, minimal residual disease in neuroblastoma can be detected with high sensitivity, even in a sample with a low gene expression level. Minimal residual disease can be found even in a sample with a tumor volume that could not be heretofore detected stably, and thus, more detailed investigation of minimal residual disease in neuroblastoma becomes possible. For example, by monitoring minimal residual disease with time, quantitative and qualitative investigation of minimal residual disease becomes possible. Furthermore, by focusing on which genetic marker of the seven genetic markers is increased or decreased, the characteristics (traits) of tumor cells for each sample can be investigated.

This allows stratification of high-risk group neuroblastoma patients based on the measured minimal residual disease, and optimization of the treatment protocol. Furthermore, relapse of a stage that could not be heretofore found can be found and treated at an early stage, and thus, in particular, the prognosis of high-risk group neuroblastoma patients can be improved.

### EXAMPLES

The present invention will be hereinafter described in detail by showing examples, although the present invention is not limited thereto. In the following examples, CRMP1 (SEQ ID NO: 1), DBH (SEQ ID NO: 2), DDC (SEQ ID NO: 3), GAP43 (SEQ ID NO: 4), ISL1 (SEQ ID NO: 5), PHOX2B (SEQ ID NO: 6), and TH (SEQ ID NO: 7) were used as MRD genetic markers, and HPRT1 (SEQ ID NO: 8) was used as a reference gene.

### Example 1

A bone marrow sample was collected from a neuroblastoma patient (at diagnosis), and the genetic markers CRMP1, DBH, DDC, GAP43, ISL1, PHOX2B, and TH were measured as follows:

1) Nucleated cells were centrifuged using the Mono-Poly Resolving Medium (manufactured by DS Pharma Biomedical) from 2 to 5 ml of the sample containing an anticoagulant (EDTA or heparin).

2) Total RNA was extracted from the obtained nucleated cells, using the TRIZOL PLUS RNA Purification Kit (manufactured by Life Technologies).

3) The extracted total RNA quality was evaluated using the RNA 6000 Nano Kit (manufactured by Agilent Technologies).

4) From 1.0 μg of the total RNA whose quality was evaluated, cDNA was synthesized using the QuantiTect Reverse Transcription Kit (manufactured by Qiagen) and diluted with TE buffer to a total volume of 80 μl.

5) The seven genetic markers were simultaneously subjected to ddPCR (digital PCR) reactions using the QX200 Droplet Digital PCR System (manufactured by Bio-Rad), and the expression level of each of the genetic markers was analyzed.

Specifically, as the primers for the genetic markers (CRMP1, DBH, DDC, GAP43, ISL1, PHOX2B, and TH) and for the reference gene (HPRT1), respectively, primers having the sequences as set forth in SEQ ID NOS: 13 to 26 and primers having the sequences as set forth in SEQ ID NOS: 27 and 28 were used; and as probes, Universal Probe Library probes (manufactured by Roche) #65 (for the CRMP1 marker), #3 (for the DBH marker), #49 (for the DDC marker), #26 (for the GAP43 marker), #66 (for the ISL1 marker), #17 (for the PHOX2B marker), #42 (for the TH marker), and #73 (for the HPRT1 marker) were used.

A total of 20 μl of a reaction mixture was prepared containing 1.0 μl of the template cDNA (corresponding to 12.5 ng of total RNA), 10 μl of 2× ddPCR Supermix for Probes (Bio-Rad), a sense primer (final 500 nM each), an anti-sense primer (final 500 nM each), and a Universal Probe Library probe (manufactured by Roche) (final 250 nM each).

Droplets of the reaction mixture were prepared using the Droplet Generator (manufactured by Bio-Rad), PCR reactions were performed for the droplets, and measurements were performed using the Droplet Reader (manufactured by Bio-Rad). The PCR reactions were performed using the Thermal Cycler Gene Amp PCR System 9700 (manufactured by Applied Biosystems) under the following conditions: a pre-reaction at 95° C. for 10 minutes, followed by 40 cycles at 94° C. for 30 seconds and 58° C. for 90 seconds per cycle, and lastly a termination reaction at 98° C. for 10 minutes.

6) From the obtained measurement results, the copy number of each MRD genetic marker in the sample (Copies per sample) was calculated from the copy number of the MRD genetic marker (Copies per well) and the copy number of the reference gene (Copies per well), based on the following equation:

(copy number of the MRD genetic marker/copy number of the reference gene)×10,000

Furthermore, mean±3SD (SD: standard deviation) obtained by measuring each of the seven genetic markers in samples (PB: peripheral blood samples, BM: bone marrow samples) from 10 healthy individuals was set as a cut-off value (threshold level) as shown below, and when the copy number of the genetic marker was lower than the cut-off value, MRD was determined as negative.

TABLE 1

| Gene Name | PB_Mean+3SD | BM_Mean+3SD |
|---|---|---|
| CRMP1 | <54 | <107 |
| DBH | <83 | <40 |
| DDC | <13 | <27 |
| GAP43 | <28 | <115 |
| ISL1 | <12 | <45 |
| PHOX2B | <12 | <19 |
| TH | <4 | <27 |

The analytical results of the bone marrow sample at diagnosis of the neuroblastoma patient are shown below.

TABLE 2

| | Copies per well | Copies per sample | |
|---|---|---|---|
| HPRT1 | 4820 | | |
| CRMP1 | 856 | 1775.9 | + |
| DBH | 3.6 | 7.5 | − |
| DDC | 66 | 136.9 | + |
| GAP43 | 2360 | 4896.3 | + |
| ISL1 | 772 | 1601.7 | + |
| PHOX2B | 1276 | 2647.3 | + |
| TH | 752 | 1560.2 | + |

As shown in the table above, in the bone marrow sample at diagnosis of the neuroblastoma patient, the values of the Copies per sample for the six genetic markers except for DBH were not lower than the cut-off values. Therefore, MRD in this sample was evaluated as positive.

### Example 2

Each of the genetic markers was measured and MRD was evaluated as in Example 1, except that a bone marrow sample in remission of a neuroblastoma patient was used as a sample. The results are shown in the table below.

TABLE 3

| | Copies per well | Copies per sample | |
|---|---|---|---|
| HPRT1 | 6060 | | |
| CRMP1 | 14 | 23.1 | − |
| DBH | 0 | 0.0 | − |
| DDC | 0 | 0.0 | − |
| GAP43 | 0 | 0.0 | − |
| ISL1 | 2.6 | 4.3 | − |
| PHOX2B | 3 | 5.0 | − |
| TH | 3.2 | 5.3 | − |

As shown in the table above, in the bone marrow sample in remission of the neuroblastoma patient, the values of the Copies per sample for all the seven genetic markers were lower than the cut-off values. Therefore, MRD in this sample was evaluated as negative.

### Example 3

Each of the genetic markers was measured and MRD was evaluated as in Example 1, except that a peripheral blood sample at diagnosis of a neuroblastoma patient was used as a sample. The results are shown in the table below.

TABLE 4

| | Copies per well | Copies per sample | |
|---|---|---|---|
| HPRT1 | 3120 | | |
| CRMP1 | 20 | 64.1 | + |
| DBH | 0 | 0.0 | − |
| DDC | 1.6 | 5.1 | − |
| GAP43 | 104 | 333.3 | + |
| ISL1 | 26 | 83.3 | + |
| PHOX2B | 26 | 83.3 | + |
| TH | 1.8 | 5.8 | + |

As shown in the table above, in the peripheral blood sample at diagnosis of the neuroblastoma patient, the values of the Copies per sample for the CRMP1, GAP43, ISL1, PHOX2B, and TH markers were not lower than the cut-off values. Therefore, MRD in this sample was evaluated as positive.

### Example 4

Each of the genetic markers was measured and MRD was evaluated as in Example 1, except that a peripheral blood sample in remission of a neuroblastoma patient was used as a sample. The results are shown in the table below.

TABLE 5

| | Copies per well | Copies per sample | |
|---|---|---|---|
| HPRT1 | 4020 | | |
| CRMP1 | 0 | 0.0 | − |
| DBH | 3.2 | 8.0 | − |
| DDC | 2.4 | 6.0 | − |
| GAP43 | 2.4 | 6.0 | − |
| ISL1 | 0 | 0.0 | − |
| PHOX2B | 1.2 | 3.0 | − |
| TH | 0 | 0.0 | − |

As shown in the table above, in the peripheral blood sample in remission of the neuroblastoma patient, the values of the Copies per sample for all the seven genetic markers were lower than the cut-off values. Therefore, MRD in this sample was evaluated as negative.

### Example 5

Each of the genetic markers was measured and MRD was evaluated as in Example 1, for bone marrow samples after the completion of one course of remission induction therapy and after the completion of five courses of remission induction therapy of a high-risk neuroblastoma patient (both samples were derived from the same patient). As a result, the values of the Copies per sample for all the seven markers were not lower than the cut-off values after the completion of one course of remission induction therapy, whereas the values of the Copies per sample for all the seven markers were lower than the cut-off values after the completion of five courses of remission induction therapy. Therefore, it was determined that this patient became MRD-negative after the completion of five courses of remission induction therapy.

Separately, each of the genetic markers was measured and MRD was evaluated as in Example 1, for a bone marrow sample and a peripheral blood sample at first examination as well as a bone marrow sample and a peripheral blood sample after the completion of five courses of remission induction therapy of a high-risk neuroblastoma patient (all samples were derived from the same patient). As a result, in the bone marrow sample at first examination, the values of the Copies per sample for six markers (specifically, CRMP1, DDC, GAP43, ISL1, PHOX2B, and TH) were not lower than the cut-off values, and in the peripheral blood sample at first examination, the values of the Copies per sample for five markers (specifically, CRMP1, GAP43, ISL1, PHOX2B, and TH) were not lower than the cut-off values, whereas in both the bone marrow sample and the peripheral blood sample after the completion of five courses of remission induction therapy, the values of the Copies per sample for all the seven markers were lower than the cut-off values. Therefore, it was determined that this patient became MRD-negative after the completion of five courses of remission induction therapy.

### Example 6

Using 11 markers, i.e., the seven markers according to the present invention (CRMP1, DBH, DDC, GAP43, ISL1, PHOX2B, and TH), and additionally other four markers reported in Non Patent Literature 6 (DCX, CHRNA3, KIF1A, and GABRB3), MRD was evaluated in a total of 252 samples, i.e., 229 bone marrow samples and 23 peripheral blood samples.

B2M reported in Non Patent Literature 6 was selected as a reference gene, and using the Thermal Cycler Gene Amp PCR System 9700 (manufactured by Applied Biosystems) as a reactor, qPCR reactions (real time PCR) were performed for these 252 samples, and the 11 markers were measured.

The primer sequences used for the qPCR reactions of these 252 samples were as follows:
primers for B2M (SEQ ID NOS: 35 and 36)
primers for CRMP1 (SEQ ID NOS: 13 and 14)
primers for DBH (SEQ ID NOS: 15 and 16)
primers for DDC (SEQ ID NOS: 17 and 18)
primers for GAP48 (SEQ ID NOS: 19 and 20)
primers for ISL1 (SEQ ID NOS: 21 and 22)
primers for PHOX2B (SEQ ID NOS: 23 and 24)
primers for TH (SEQ ID NOS: 25 and 26)

primers for DCX
5′-catccccaacacctcagaag-3′ (sense)
5′-ggaggttccgtttgctga-3′ (anti-sense)
primers for CHRNA3
5′-tgaaatggaacccctctgac-3′ (sense)
5′-ggaaatccccaacagcatt-3′ (anti-sense)
primers for KIF1A
5′-cttggcgacatcactgacat-3′ (sense)
5′-gctggacagggctgagag-3′ (anti-sense)
primers for GABRB3
5′-gggtgtccttctggatcaatta-3′ (sense)
5′-ttgtcagcacagttgtgatcc-3′ (anti-sense)

Furthermore, as reported in Non Patent Literature 6, cut-off values (threshold levels) were set as shown below, and when the expression levels of the markers were lower than the cut-off values, the markers were determined as negative.

TABLE 6

| Gene name | PB_% dilution | BM_% dilution |
|---|---|---|
| CHRNA3 | <0.032 | <0.413 |
| CRMP1 | <0.032 | <0.207 |
| DBH | <0.032 | <0.032 |
| DCX | <0.160 | <5.598 |
| DDC | <0.032 | <0.051 |
| GABRB3 | <0.032 | <1.059 |
| GAP43 | <0.032 | <0.694 |
| ISL1 | <0.160 | <0.160 |
| KIF1A | <0.032 | <0.351 |
| PHOX2B | <0.032 | <0.032 |
| TH | <0.160 | <0.160 |

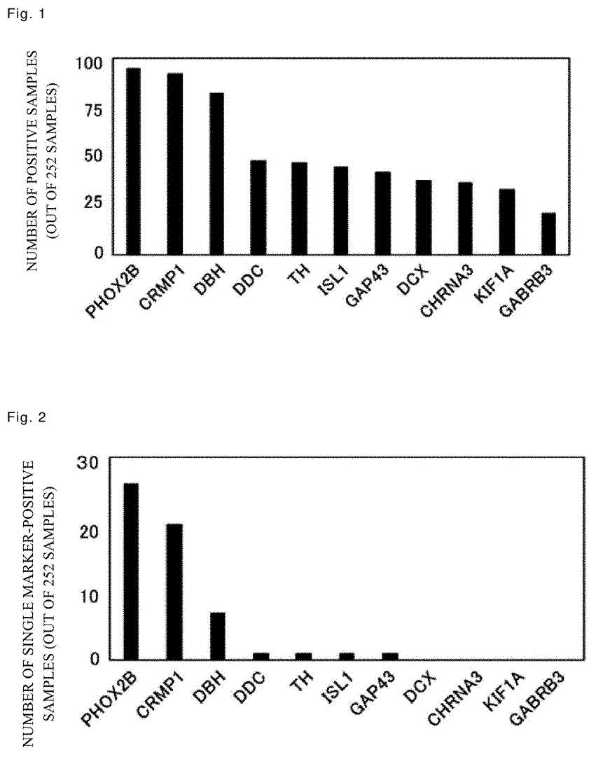

FIG. 1 shows the number of samples, out of the 252 samples, in which any of the seven markers according to the present invention (CRMP1, DBH, DDC, GAP43, ISL1, PHOX2B, and TH) and the other four markers (DCX, CHRNA3, KIF1A, and GABRB3) became positive. In FIG. 1, for example, when both CRMP1 and DCX were detected in one sample, CRMP1 was counted as one positive, and DCX was counted as one positive. Furthermore, FIG. 2 shows the number of samples, out of the 252 samples, in which any of the seven markers according to the present invention and the other four markers alone became positive (single marker-positive).

Although a considerable number of the other four markers were detected as shown in FIG. 1, only these other four markers did not have detection performance as single markers, as shown in FIG. 2. These four markers were detected together with the seven markers according to the present invention, and even if these markers per se were not measured, there was no influence on the number of false negatives, and there was no substantial influence on the screening sensitivity. This shows that the seven markers according to the present invention were able to reduce the number of clinically applicable markers, while reducing false negatives.

### Example 7

As in Example 1, using ddPCR, each of the genetic markers was measured, and MRD in bone marrow samples or peripheral blood samples from neuroblastoma patients was evaluated. The results are shown in the table below.

TABLE 7
Bone Marrow Sample from Patient Undergoing Initial
Treatment (PHOX2B-Positive)

| Gene name | Copies per sample | |
|---|---|---|
| CRMP1 | 35.1 | − |
| DBH | 8.8 | − |
| DDC | 6.1 | − |
| GAP43 | 30.7 | − |
| ISL1 | 10.1 | − |
| PHOX2B | 19.7 | + |
| TH | 0.0 | − |

TABLE 8
Bone Marrow Sample from Patient Undergoing Relapse
Treatment (CRMP1-Positive)

| Gene name | Copies per sample | |
|---|---|---|
| CRMP1 | 205.3 | + |
| DBH | 4.0 | − |
| DDC | 0.0 | − |
| GAP43 | 0.0 | − |
| ISL1 | 4.0 | − |
| PHOX2B | 0.0 | − |
| TH | 0.0 | − |

TABLE 9
Bone Marrow Sample from Patient Undergoing Initial
Treatment (DBH-Positive)

| Gene name | Copies per sample | |
|---|---|---|
| CRMP1 | 31.6 | − |
| DBH | 82.9 | + |
| DDC | 0.0 | − |
| GAP43 | 2.6 | − |
| ISL1 | 2.4 | − |
| PHOX2B | 1.2 | − |
| TH | 1.4 | − |

TABLE 10
Peripheral Blood Sample from Patient Undergoing Relapse
Treatment (DDC-Positive)

| Gene name | Copies per sample | |
|---|---|---|
| CRMP1 | 29.3 | − |
| DBH | 0.0 | − |
| DDC | 27.1 | + |
| GAP43 | 0.0 | − |
| ISL1 | 0.0 | − |
| PHOX2B | 8.8 | − |
| TH | 0.0 | − |

TABLE 11
Peripheral Blood Sample from Patient Undergoing Initial
Treatment (TH-Positive)

| Gene name | Copies per sample | |
|---|---|---|
| CRMP1 | 0.0 | − |
| DBH | 3.1 | − |
| DDC | 0.0 | − |
| GAP43 | 8.8 | − |
| ISL1 | 0.0 | − |
| PHOX2B | 3.1 | − |
| TH | 5.7 | + |

TABLE 12
Peripheral Blood Sample from Patient Undergoing Initial
Treatment (ISL1-Positive)

| Gene name | Copies per sample | |
|---|---|---|
| CRMP1 | 30.1 | − |
| DBH | 4.5 | − |
| DDC | 4.5 | − |
| GAP43 | 8.3 | − |
| ISL1 | 12.8 | + |
| PHOX2B | 0.0 | − |
| TH | 0.0 | − |

TABLE 13
Bone Marrow Sample from Patient Undergoing Initial
Treatment (GAP43-Positive)

| Gene name | Copies per sample | |
|---|---|---|
| CRMP1 | 26.8 | − |
| DBH | 5.0 | − |
| DDC | 0.0 | − |
| GAP43 | 130.4 | + |
| ISL1 | 15.7 | − |
| PHOX2B | 0.0 | − |
| TH | 0.0 | − |

As shown in the tables above, also when ddPCR was used, all the seven markers according to the present invention (CRMP1, DBH, DDC, GAP43, ISL1, PHOX2B, and TH) were demonstrated to have the ability to detect MRD as single markers.

### Example 7

The BE(2)-C neuroblastoma cell line (manufactured by American Type Culture Collection) was serially diluted in normal bone marrow cells or normal peripheral blood cells, and each of the genetic markers was measured using ddPCR as in Example 1 to study the detection limit. Similarly, each of the genetic markers was measured using qPCR as in Example 6 to study the detection limit. These results are shown in Table 14. In the table, BM represents the specimen diluted with normal bone marrow cells; PB represents the specimen diluted with normal peripheral blood cells; the numerical values represent the dilution ratios at detection limits; and the numerical values within parentheses represent the sensitivity of ddPCR (digital PCR) relative to qPCR (real time PCR). As shown in Table 14, because ddPCR has higher sensitivity, MRD can be detected with higher sensitivity even in samples with lower expression.

TABLE 14

| | BE(2)-C | | | |
|---|---|---|---|---|
| MRD | BM | | PB | |
| marker | qPCR | ddPCR (Sensitivity) | qPCR | ddPCR (Sensitivity) |
| CRMP1 | 1/100 | 1/10000 (100-fold) | 1/1000 | 1/10000 (10-fold) |
| DBH | 1/1000 | 1/10000 (10-fold) | 1/1000 | 1/10000 (10-fold) |
| DDC | 1/1000 | 1/10000 (10-fold) | 1/1000 | 1/10000 (10-fold) |
| GAP43 | 1/100 | 1/1000 (10-fold) | 1/1000 | 1/10000 (10-fold) |
| ISL1 | 1/100 | 1/10000 (100-fold) | 1/100 | 1/10000 (100-fold) |
| PHOX2B | 1/1000 | 1/10000 (10-fold) | 1/1000 | 1/10000 (10-fold) |
| TH | 1/100 | 1/10000 (100-fold) | 1/100 | 1/100000 |
| | | | | (1000-fold) |

### Test Example

In 10 bone marrow (BM) samples and 10 peripheral blood (PB) samples collected from 10 healthy individuals, the reference genes GUSB, HMBS, HPRT1, and TBP were amplified using ddPCR as in Example 1. Table 15 shows the copy numbers of these reference gene markers.

TABLE 15

| GUSB | | HMBS | | HPRT1 | | TBP | |
|---|---|---|---|---|---|---|---|
| BM | PB | BM | PB | BM | PB | BM | PB |
| 2120 | 1680 | 22420 | 918 | 3160 | 2380 | 1194 | 1380 |
| 1540 | 2640 | 2340 | 966 | 3420 | 3860 | 486 | 2980 |
| 1420 | 3300 | 2500 | 1478 | 1540 | 3520 | 488 | 2820 |
| 1860 | 2080 | 15140 | 1064 | 4160 | 3640 | 1040 | 1980 |
| 1860 | 2740 | 44400 | 1016 | 8020 | 4020 | 1260 | 2420 |
| 1580 | 2880 | 14640 | 1114 | 3540 | 4160 | 1600 | 2140 |
| 3100 | 2580 | 19340 | 1558 | 7920 | 4420 | 1720 | 2720 |
| 92 | 2500 | 7560 | 2480 | 2040 | 3600 | 268 | 2460 |
| 1580 | 1900 | 7800 | 840 | 4540 | 3160 | 1214 | 2020 |
| 2240 | 3580 | 18640 | 928 | 5020 | 5540 | 1620 | 3460 |

As shown in Table 15, among these reference genes, only HPRT1 showed low variations in expression level among the samples, as well as low variations in expression level between the bone marrow samples and the peripheral blood samples, and was stably expressed. It was also confirmed that B2M used as a reference gene for qPCR (real time PCR) in Example 6, when applied to a ddPCR (digital PCR) assay system, is overexpressed and saturated, and thus, is unable to quantify expression levels. That is, HPRT1 was demonstrated to be a reference gene that is expressed at low level and is thus suitable for digital PCR, and is particularly suitable for accurately detecting minimal residual disease in neuroblastoma by digital PCR, both in bone marrow samples and peripheral blood samples.

While preferred embodiments of the present invention are as described above, the present invention is not limited to the foregoing embodiments, and various modifications may be made without departing from the gist of the present invention.

### Sequence Listing Free Text

SEQ ID NOS: 13 to 36 are primers.

1. A reagent used for evaluating minimal residual disease in neuroblastoma, comprising a primer pair capable of amplifying genetic markers consisting of CRMP1, DBH, DDC, GAP43, ISL1, PHOX2B, and TH, by a nucleic acid amplification method.

2. The reagent according to claim 1, further comprising a primer pair capable of amplifying at least any of HPRT1, HMBS, GUSB, TBP, and B2M as reference genes, by the nucleic acid amplification method.

3. The reagent according to claim 1, further comprising a primer pair capable of amplifying at least any of HPRT1, HMBS, GUSB, and TBP as reference genes, by the nucleic acid amplification method.

4. The reagent according to claim 1, further comprising a primer pair capable of amplifying a reference gene consisting of HPRT1, by the nucleic acid amplification method.

5. The reagent according to claim 1, further comprising a probe capable of hybridizing with any of the genetic markers under stringent conditions.

6. The reagent according to claim 5, further comprising, when the reagent further comprises a primer pair capable of amplifying a reference gene by the nucleic acid amplification method, a probe capable of hybridizing with the reference gene under stringent conditions.

7. A method of analyzing a biological specimen comprising:
a measurement step of measuring an expression levels of genetic markers consisting of CRMP1, DBH, DDC, GAP43, ISL1, PHOX2B, and TH in the biological specimen by a nucleic acid amplification method, using the reagent according to claim 1, wherein
the expression levels are correlated with a level of occurrence of minimal residual disease in neuroblastoma.

8. The method of analyzing a biological specimen according to claim 7, comprising an evaluation step of evaluating whether or not the expression level of at least one of the genetic markers is not lower than a threshold level.

9. The method of analyzing a biological specimen according to claim 8, wherein in the evaluation step, when the expression level of at least one of the genetic markers is not lower than the threshold level, minimal residual disease in neuroblastoma in the biological specimen is determined as positive.

10. The method of analyzing a biological specimen according to claim 7, wherein the nucleic acid amplification method in the measurement step is digital PCR.

11. The method of analyzing a biological specimen according to claim 10, wherein in the measurement step, the genetic markers are simultaneously subjected to nucleic acid amplification, using an identical nucleic acid amplification apparatus

12. The method according to claim 7, wherein the biological specimen is a bone marrow sample.


SEQUENCE LISTING (36 sequences)

SEQ ID NO: 1 - DNA, 3155 bp, human
gcaccgaggcgcggagcgcggcgcacggcggtgcccagggccgcgggcgggcaggcgggagcgcggtgggtggccggccttggacgggtatggcggaccggcggcgcgcgtggaacaccgaggacgacctgcccgtgtacctggcgcggccgggcagcgcggcgcagaccccgcgccagaagtacggcggcatgttcgccgcggtggagggcgcctacgagaacaagaccatcgacttcgacgcctacagtgtgggccgccgcggctcggcgcgcacgccgcgcagcgctggccggcccgacgcggtcgggctgccagggccgggaggcagcgaggacacggccagcgacgtgagcgagccctcgggctccgcggtcagctctcccggagagcgcgacgagcggccgccgacgctgcgcatccgccgccccgcgccccgcgacctgcccctcggccgggacaatggccagagtgaccgactcctcatcaaaggtggacggatcatcaacgatgaccaatccctttatgctgacgtctacctggaggatggacttatcaaacaaataggagagaacttaatcgttcctggtggagtgaagaccattgaagccaacgggcggatggttattcccggaggtattgatgtcaacacgtacctgcagaagccctcccaggggatgactgcggctgatgacttcttccaagggaccagggcggcactggtgggcgggaccacgatgatcattgaccatgttgttcctgaacctgggtccagcctactgacctctttcgagaagtggcacgaagcagctgacaccaaatcctgctgtgattactccctccacgtggacatcacaagctggtacgatggcgttcgggaggagctggaggtgctggtgcaggacaaaggcgtcaattccttccaagtctacatggcctataaggatgtctaccaaatgtccgacagccagctctatgaagcctttaccttccttaagggcctgggagctgtgatcttggtccatgcagaaaatggagatttgatagctcaggaacaaaagcggatcctggagatgggcatcacgggtcccgagggccatgccctgagcagacctgaagagctggaggccgaggcggtgttccgggccatcaccattgcgggccggatcaactgccctgtgtacatcaccaaggtcatgagcaagagtgcagccgacatcatcgctctggccaggaagaaagggcccctagtttttggagagcccattgccgccagcctggggaccgatggcacccattactggagcaagaactgggccaaggctgcggcgttcgtgacttcccctcccctgagcccggaccctaccacgcccgactacttgacctccctactggcctgtggggacttgcaggtcacaggcagcggccactgtccctacagcactgcccagaaggcggtgggcaaggacaactttaccctgatccccgagggtgtcaacgggatagaggagcggatgacggtcgtctgggacaaggcggtggctactggcaaaatggatgagaaccagtttgtcgctgtcaccagcaccaatgcagccaagatctttaacctgtacccaaggaaagggcggattgccgtgggctcggatgccgacgtggtcatctgggaccccgacaagttgaagaccataacagccaaaagtcacaagtcggcggtggagtacaacatcttcgagggtatggagtgccacggctccccactagtggtcatcagccagggcaagatcgtctttgaagacggaaacatcaacgtcaacaagggcatgggccgcttcattccgcggaaggcgttcccggagcacctgtaccagcgcgtcaaaatcaggaataaggtttttggattgcaaggggtttccaggggcatgtatgacggtcctgtgtacgaggtaccagctacacccaaatatgcaactcccgctccttcagccaaatcttcgccttctaaacaccagcccccacccatcagaaacctccaccagtccaacttcagcttatcaggtgcccagatagatgacaacaatcccaggcgcaccggccaccgcatcgtggcgccccctggtggccgctccaacatcaccagcctcggttgaacgtggatgcgcggaggagctagcctgaaggattctgggaatcatgtccatcccttttcctgtcagtgtttttgaaacccacagttttagttggtgctgatggagggagggggaagtcgaaggatgctctttcccttttctgtttaggaagaagtggtactagtgtggtgtgtttgcttggaaattccttgccccacagttgtgttcatgctgaatccacctcggagcatggtgttttcattcccccttcctagtgaaccacaggttttagcattgtcttgttctgtcccttccacttctaactccactggctccatgattctctgagtggtggttcctttgcaccctgtagatgttctaggatagttgatgcatgttactaaattacgtatgcaagtctgtgagtgcgtctgaggggacatcgccaaggactgactgagacacgatgccgagacctcaagccctgaggggcagtcccaaaacccttacagtgaagatgtttactcattgcccccacctctggtccacactagaaagaagctcgccccacctccacctgtgagatccgtgaattctcggaatggcaggggaagccttgcactaggttgcagagaagcatcctccacatcctgtgtcagaaaccctggtctccgtggcacttgtaactcaccgtgctgtcttctggtctgtgtgtgttcttcaagccagctctaggcttcaggccgagccaggttcacactcagaaagaggtctccccatccccattcggggctgacgatggggggctgatggctgcccctgcgtggcctgagtcctggtccctctgaggcagttgacggggcagtcagatttttaaagttttgtacaaagttttcctttgtaatcactcccatttttacttaacaaccaacttgttgtggctcttatttctgaattcaaagcttgtgaaaaaataaagaaaatgaactgcccactgacctgactagggcactttcaaaaaaaaaa

SEQ ID NO: 2 - DNA, 2812 bp, human
accccagccatgcccgccctcagtcgctgggccagcctgcccggccccagcatgcgggaggcagccttcatgtacagcacagcagtggccatcttcctggtcatcctggtggccgcactgcagggctcggctccccgtgagagccccctcccctatcacatccccctggacccggaggggtccctggagctctcatggaatgtcagctacacccaggaggccatccatttccagctcctggtgcggaggctcaaggctggcgtcctgtttgggatgtccgaccgtggcgagcttgagaacgcagatctcgtggtgctctggaccgatggggacactgcctattttgcggacgcctggagtgaccagaaggggcagatccacctggatccccagcaggactaccagctgctgcaggtgcagaggaccccagaaggcctgaccctgcttttcaagaggccctttggcacctgcgaccccaaggattacctcattgaagacggcactgtccacttggtctacgggatcctggaggagccgttccggtcactggaggccatcaacggctcgggcctgcagatggggctgcagagggtgcagctcctgaagcccaatatccccgaaccggagttgccctcagacgcgtgcaccatggaggtccaagctcccaatatccagatccccagccaggagaccacgtactggtgctacattaaggagcttccaaagggcttctctcggcaccacattatcaagtacgagcccatcgtcaccaagggcaatgaggcccttgtccaccacatggaagtcttccagtgcgcccccgagatggacagcgtcccccacttcagcgggccctgcgactccaagatgaaacccgaccgcctcaactactgccgccacgtgctggccgcctgggccctgggtgccaaggcattttactacccagaggaagccggccttgccttcgggggtccagggtcctccagatatctccgcctggaagttcactaccacaacccactggtgatagaaggacgaaacgactcctcaggcatccgcttgtactacacagccaagctgcggcgcttcaacgcggggatcatggagctgggactggtgtacacgccagtgatggccattccaccacgggagaccgccttcatcctcactggctactgcacggacaagtgcacccagctggcactgcctccctccgggatccacatcttcgcctctcagctccacacacacctgactgggagaaaggtggtcacagtgctggtccgggacggccgggagtgggagatcgtgaaccaggacaatcactacagccctcacttccaggagatccgcatgttgaagaaggtcgtgtcggtccatccgggagatgtgctcatcacctcctgcacgtacaacacagaagaccgggagctggccacagtggggggcttcgggatcctggaggagatgtgtgtcaactacgtgcactactacccccagacgcagctggagctctgcaagagcgctgtggacgccggcttcctgcagaagtacttccacctcatcaacaggttcaacaacgaggatgtctgcacctgccctcaggcgtccgtgtctcagcagttcacctctgttccctggaactccttcaaccgcgacgtactgaaggccctgtacagcttcgcgcccatctccatgcactgcaacaagtcctcagccgtccgcttccagggtgaatggaacctgcagcccctgcccaaggtcatctccacactggaagagcccaccccacagtgccccaccagccagggccgaagccctgctggccccaccgttgtcagcattggtgggggcaaaggctgaggggggacctactcctccccctcctccatgctgtccctgtgggctcacaccggcactgtgcactctactctgcgacgatccccatggaacagccctgcacgcccaggatgaaggggccagaccacgcccctgcctgagaccacggtccaatccagccttcttcccccagggtcccctgcatggctgagagggtgtgggtgccctgttgacctaccctggaccgagtggaccacgacctcgtccatttaaacccggctgactcagtgcagggacagcctgcacagtggtccagggtccagccctccgccagccctgttccgcctcactgggtgtggcctggcttctgggacaggcaccatgctgggccggggtgtggaatcaccgggaacgcccccgcccccgccccgctgctcccggtgtgcagcgggtgcgggtgccgcttaaacatttccctgctgagtggctcgtgtttcacagtgggcggcttccctgcgacggaggcaggaccaggcatttagctagttagagactcgcctgggaaattgctccattcctgagtaaacagatattttcgcccacctaaagggaagccctgacaacaactatcaccaaaagacgaggcggcaaagatccagcggggcttctgggcgccggttccacgtggggtggaattattagcaccagcttgcttctctgccggtggggccagcgctgaacagaccggggtggagtcagggctgtgctttccgcgtggttctgccacttagggagtgtgccttgggcgggccatttcacattcctgaccctcacttttctcatctgtaaaaccaggctgatgccgtgcgggctaatgagccaataaagctcacacttgggctggcacccactggaggtgggaaaaaaaaaaaaaaaaaaaaaaaaaaaaaaaaaaaaaaaaaaaaaaaaaaaa

SEQ ID NO: 3 - DNA, 1975 bp, human
aactgtcactgtggagaggagagagagaggacagagagcaagtcactcccggctgcctttttcacctctgacagagcccagacaccatgaacgcaagtgaattccgaaggagagggaaggagatggtggattacgtggccaactacatggaaggcattgagggacgccaggtctaccctgacgtggagcccgggtacctgcggccgctgatccctgccgctgcccctcaggagccagacacgtttgaggacatcatcaacgacgttgagaagataatcatgcctggggtgacgcactggcacagcccctacttcttcgcctacttccccactgccagctcgtacccggccatgcttgcggacatgctgtgcggggccattggctgcatcggcttctcctgggcggcaagcccagcatgcacagagctggagactgtgatgatggactggctcgggaagatgctggaactaccaaaggcatttttgaatgagaaagctggagaagggggaggagtgatccagggaagtgccagtgaagccaccctggtggccctgctggccgctcggaccaaagtgatccatcggctgcaggcagcgtccccagagctcacacaggccgctatcatggagaagctggtggcttactcatccgatcaggcacactcctcagtggaaagagctgggttaattggtggagtgaaattaaaagccatcccctcagatggcaacttcgccatgcgtgcgtctgccctgcaggaagccctggagagagacaaagcggctggcctgattcctttctttatggttgccaccctggggaccacaacatgctgctcctttgacaatctcttagaagtcggtcctatctgcaacaaggaagacatatggctgcacgttgatgcagcctacgcaggcagtgcattcatctgccctgagttccggcaccttctgaatggagtggagtttgcagattcattcaactttaatccccacaaatggctattggtgaattttgactgttctgccatgtgggtgaaaaagagaacagacttaacgggagcctttagactggaccccacttacctgaagcacagccatcaggattcagggcttatcactgactaccggcattggcagataccactgggcagaagatttcgctctttgaaaatgtggtttgtatttaggatgtatggagtcaaaggactgcaggcttatatccgcaagcatgtccagctgtcccatgagtttgagtcactggtgcgccaggatccccgctttgaaatctgtgtggaagtcattctggggcttgtctgctttcggctaaagggttccaacaaagtgaatgaagctcttctgcaaagaataaacagtgccaaaaaaatccacttggttccatgtcacctcagggacaagtttgtcctgcgctttgccatctgttctcgcacggtggaatctgcccatgtgcagcgggcctgggaacacatcaaagagctggcggccgacgtgctgcgagcagagagggagtaggagtgaagccagctgcaggaatcaaaaattgaagagagatatatctgaaaactggaataagaagcaaataaatatcatcctgccttcatggaactcagctgtctgtggcttcccatgtctttctccaaagttatccagagggttgtgattttgtctgcttagtatctcatcaacaaagaaatattatttgctaattaaaaagttaatcttcatggccatagcttttattcattagctgtgatttttgttgattaaaacattatagattttcatgttcttgcagtcatcagaagtggtaggaaagcctcactgatatattttccagggcaatcaatgttcacgcaacttgaaattatatctgtggtcttcaaattgtcttttgtcatgtggctaaatgcctaataaacaattcaagtgaaatactaaaaaaaaaaaaaaaaaaaaaa

SEQ ID NO: 4 - DNA, 2143 bp, human
actgaaggctagagaacaattccgagaaagagacggagagagagggaagaaaaagacagatagatagatattggggggaaggagaaaaaaggagaagagagggaagagaggacagcggagagagagcaccagagagagagggagagagagagagagcgctagagagagggagcgagcatgtgcgatgagcaatagctgtggaccttacagttgctgctaactgccctggtgtgtgtgagggagagagagggagggagggagagagagcgcgctagcgcgagagagcgagtgagcaagcgagcagaaaagaggtggagagggggggaataagaaagagagagaaggaaaggagagaaggcaggaagaaggcaagggacgagacaaccatgctgtgctgtatgagaagaaccaaacagaattaaaagggaacctggtctctgggttgttttcaacatctcaagtgtgaattttccctgtcaaaatcttcacaaggaaaatgagtcacagcatcacctgggtgacgaggtcataacacctcagcccttgcttaaaaaattttatttctacttttctattgtaaagagatctcaaaacaggaagataaaattggactgacagctctacagcctagtcttttagacagtgaactaggccagcattggcagacactggcgatgacaaagtcctgctctgaattatgccaccccgcactccactttttaccttgcctgggaggcttgaggaaaaatcttcagagagcagttcgacctagtccttattcacttggcttcttgactttctggatttcaagggttgaaaaaaatgatgacgaccaaaagattgaacaagatggtatcaaaccagaagataaagctcataaggccgcaaccaaaattcaggctagcttccgtggacacataacaaggaaaaagctcaaaggagagaagaaggatgatgtccaagctgctgaggctgaagctaataagaaggatgaagcccctgttgccgatggggtggagaagaagggagaaggcaccactactgccgaagcagccccagccactggctccaagcctgatgagcccggcaaagcaggagaaactccttccgaggagaagaagggggagggtgatgctgccacagagcaggcagccccccaggctcctgcatcctcagaggagaaggccggctcagctgagacagaaagtgccactaaagcttccactgataactcgccgtcctccaaggctgaagatgccccagccaaggaggagcctaaacaagccgatgtgcctgctgctgtcactgctgctgctgccaccacccctgccgcagaggatgctgctgccaaggcaacagcccagcctccaacggagactggggagagcagccaagctgaagagaacatagaagctgtagatgaaaccaaacctaaggaaagtgcccggcaggacgagggtaaagaagaggaacctgaggctgaccaagaacatgcctgaactctaagaaatggctttccacatccccaccctcccctctcctgagcctgtctctccctaccctcttctcagctccactctgaagtcccttcctgtcctgctcacgtctgtgagtctgtcctttcccacccactagccctctttctctctgtgtggcaaacatttaaaaaaaaaaaaaaaaagcaggaaagatcccaagtcaaacagtgtggcttaaacattttttgtttcttggtgttgttatggcaagtttttggtaatgatgattcaatcattttgggaaattcttgcactgtatccaagttatttgatctggtgcgtgtggccctgtgggagtccactttcctctctctctctctctctgttccaagtgtgtgtgcaatgttccgttcatctgaggagtccaaaatatcgagtgaattcaaaatcatttttgttttcctccttttcaatgtgatggaatgaacaaaaaggaaaaaattcaaaaaacccagtttgttttaaaaataaataaataaagcaaatgtgccaattagcgtaaacttgcggctctaaggctcctttttcaacccgaatattaataaatcatgagagtaatcaaggtcaaaaaaaaaaaaaaaaaaaaaaaaaaaaaaaaaaaaaaa

SEQ ID NO: 5 - DNA, 2729 bp, human
gaaggaagaggaagaggaggagagggaggccagagccagaacagcccggcagcccgagcttcgggggagaacggcctgagccccgagcaagttgcctcgggagccctaatcctctcccgctggctcgccgagcggtcagtggcgctcagcggcggcgaggctgaaatatgataatcagaacagctgcgccgcgcgccctgcagccaatgggcgcggcgctcgcctgacgtccccgcgcgctgcgtcagaccaatggcgatggagctgagttggagcagagaagtttgagtaagagataaggaagagaggtgcccgagccgcgccgagtctgccgccgccgcagcgcctccgctccgccaactccgccggcttaaattggactcctagatccgcgagggcgcggcgcagccgagcagcggctctttcagcattggcaaccccaggggccaatatttcccacttagccacagctccagcatcctctctgtgggctgttcaccaactgtacaaccaccatttcactgtggacattactccctcttacagatatgggagacatgggagatccaccaaaaaaaaaacgtctgatttccctatgtgttggttgcggcaatcagattcacgatcagtatattctgagggtttctccggatttggaatggcatgcggcatgtttgaaatgtgcggagtgtaatcagtatttggacgagagctgtacatgctttgttagggatgggaaaacctactgtaaaagagattatatcaggttgtacgggatcaaatgcgccaagtgcagcatcggcttcagcaagaacgacttcgtgatgcgtgcccgctccaaggtgtatcacatcgagtgtttccgctgtgtggcctgcagccgccagctcatccctggggacgaatttgcgcttcgggaggacggtctcttctgccgagcagaccacgatgtggtggagagggccagtctaggcgctggcgacccgctcagtcccctgcatccagcgcggccactgcaaatggcagcggagcccatctccgccaggcagccagccctgcggccccacgtccacaagcagccggagaagaccacccgcgtgcggactgtgctgaacgagaagcagctgcacaccttgcggacctgctacgccgcaaacccgcggccagatgcgctcatgaaggagcaactggtagagatgacgggcctcagtccccgtgtgatccgggtctggtttcaaaacaagcggtgcaaggacaagaagcgaagcatcatgatgaagcaactccagcagcagcagcccaatgacaaaactaatatccaggggatgacaggaactcccatggtggctgccagtccagagagacacgacggtggcttacaggctaacccagtggaagtacaaagttaccagccaccttggaaagtactgagcgacttcgccttgcagagtgacatagatcagcctgcttttcagcaactggtcaatttttcagaaggaggaccgggctctaattccactggcagtgaagtagcatcaatgtcctctcaacttccagatacacctaacagcatggtagccagtcctattgaggcatgaggaacattcattctgtattttttttccctgttggagaaagtgggaaattataatgtcgaactctgaaacaaaagtatttaacgacccagtcaatgaaaactgaatcaagaaatgaatgctccatgaaatgcacgaagtctgttttaatgacaaggtgatatggtagcaacactgtgaagacaatcatgggattttactagaattaaacaacaaacaaaacgcaaaacccagtatatgctattcaatgatcttagaagtactgaaaaaaaaagacgtttttaaaacgtagaggatttatattcaaggatctcaaagaaagcattttcatttcactgcacatctagagaaaaacaaaaatagaaaattttctagtccatcctaatctgaatggtgctgtttctatattggtcattgccttgccaaacaggagctccagcaaaagcgcaggaagagagactggcctccttggctgaaagagtcctttcaggaaggtggagctgcattggtttgatatgtttaaagttgactttaacaaggggttaattgaaatcctgggtctcttggcctgtcctgtagctggtttattttttactttgccccctccccactttttttgagatccatcctttatcaagaagtctgaagcgactataaaggtttttgaattcagatttaaaaaccaacttataaagcattgcaacaaggttacctctattttgccacaagcgtctcgggattgtgtttgacttgtgtctgtccaagaacttttcccccaaagatgtgtatagttattggttaaaatgactgttttctctctctatggaaataaaaaggaaaaaaaaaaaggaaactttttttgtttgctcttgcattgcaaaaattataaagtaatttattatttattgtcggaagacttgccacttttcatgtcatttgacattttttgtttgctgaagtgaaaaaaaaagataaaggttgtacggtggtctttgaattatatgtctaattctatgtgttttgtctttttcttaaatattatgtgaaatcaaagcgccatatgtagaattatatcttcaggactatttcactaataaacatttggcatagataaataaataaaaaaaaaaaaaa

SEQ ID NO: 6 - DNA, 3033 bp, human
ttaaattctaattagagatgcaggaatcaatgatagggaggttggacagctcagttccccagtgccagcccaatagacggatgagttattgtcatgtaaaaagcgccagcaataagaccaaccgctttgctattgtccaagtggaaagagccaagtttattatgaggactatatgctctagagacctcagacaaggcatctcataggaggctttttcataaaactaggctctgctggtagtaaggaggccagtttggaggcaggcgttgagctgtgcacatctccccactccagccaccttctccatatccatcttttatttcatttttccacttggctgagccatccagaaccttttcaatgtataaaatggaatattcttacctcaattcctctgcctacgagtcctgtatggctgggatggacacctcgagcctggcttcagcctatgctgacttcagttcctgcagccaggccagtggcttccagtataacccgataaggaccacttttggggccacgtccggctgcccttccctcacgccgggatcctgcagcctgggcaccctcagggaccaccagagcagtccgtacgccgcagttccttacaaactcttcacggaccacggcggcctcaacgagaagcgcaagcagcggcgcatccgcaccactttcaccagtgcccagctcaaagagctggaaagggtcttcgcggagactcactaccccgacatctacactcgggaggagctggccctgaagatcgacctcacagaggcgcgagtccaggtgtggttccagaaccgccgcgccaagtttcgcaagcaggagcgcgcagcggcagccgcagcggccgcggccaagaacggctcctcgggcaaaaagtctgactcttccagggacgacgagagcaaagaggccaagagcactgacccggacagcactgggggcccaggtcccaatcccaaccccacccccagctgcggggcgaatggaggcggcggcggcgggcccagcccggctggagctccgggggcggcggggcccgggggcccgggaggcgaacccggcaagggcggcgcagcagcagcggcggcggccgcggcagcggcggcggcggcagcggcagcggcggcagctggaggcctggctgcggctgggggccctggacaaggctgggctcccggccccggccccatcacctccatcccggattcgcttgggggtcccttcgccagcgtcctatcttcgctccaaagacccaacggtgccaaagccgccttagtgaagagcagtatgttctgatctggaatcctgcggcggcggcggcggcggcgacagcgggcgagccagggcccgggcgggcgagtgggcgagcgggtaggcccaaggctattgtcgtcgctgctgccatggctttttcattgagggcctaaagtaatcgcgctaagaataaagggaaaacggcgtcgccctcatttcaaccccactcctacccccttcctcaacccccaaacaaaacaaacaaacttccctggcttcgcacctgcctggggcctcgcagcggggccagggctccgcctgctgatcgggggttgtgagcagcgcggcctggacgcggggcactctcagggggctgtgtctgcgtgtcagtttgtgtctgtctcggggaatgtgtgtctgtggcccaagcaggtgacaggaagagatggggggcctcaaccaacttagtgacttgtttagaaaaaaaagacaaaaaagtaaaaataaaaacaaaaaagttggaaggcagaaaccattaaaaaacaaaaagccaacaacccagaaaggtttaaaaaacataaggaaaaaaaagacaaattaaaggaggggctaggggagaagctgcagctggagctgaaggctcgatcttgtgaacccctaaatccgctccctcctaacagcacggattctcttggggctcttcttcagggaagagtagggacgccgttccagccccccttcctatcgtgtccttgggttcgggtcactgcggcgacgacttgctcagactgtcccggcggccggagtgactttctcgcacccccttgcctgtcccacctcgctgaacaccatcccgccattagcgcatcggaaccccacacagttgcaactcccaaccccgaatctttgcagccgttcggccctgaaagatgccctatccatgagatgccttttcatctgcaaactctgcaaaatgtgtctcatgtttcgcaactctttttttccccctcgctcccgcctaccccgtcggcattttcttcttccaccagcttttactgaactttttggcactgctttggattggggtcaattgcagtccacgtaactggctgcagagaaatctaccgagcaaggaaaaggcacacacacacgtttgcaggggtgtctcggtttgcatttctgttggaatgatccgaactggactcacatcctgtatggtggatggactgtatattgagggttccattcttcgcgcagtttagacatctctgttttgattctttgttgttgtttttattttaaaaggcacaaactctagatattagttgaatgttgaggctttaactttttcggtgtctttctacaactgtgttctgtgactcaattgtatcgtgttaatatcagtgcagactgtctcctctacgtgaccgtataatgtttttctcttcttgtagtctctatggcgtgtctttatggtgtaataaggttctcacgggttcaatcttttgtgtttagagaggccacggttcagacaatggtatatatttttgttatcaggtgcatgtctgtctgatttctttttttttcctgttggactatgtttgtgaacataattgtcataagttatgtttcagatttttgaatttatttatatgtgttataatgaatgcttctatttaaaagggaaatatttctacatgtgcatatagttttccaagagtgtaccattaacttgattgttgataataaaaacaaaaagcaagtctaaaa

SEQ ID NO: 7 - DNA, 1910 bp, human
cggacctccacactgagccatgcccacccccgacgccaccacgccacaggccaagggcttccgcagggccgtgtctgagctggacgccaagcaggcagaggccatcatggtaagagggcagggcgccccggggcccagcctcacaggctctccgtggcctggaactgcagccccagctgcatcctacacccccaccccaaggtccccgcggttcattgggcgcaggcagagcctcatcgaggacgcccgcaaggagcgggaggcggcggtggcagcagcggccgctgcagtcccctcggagcccggggaccccctggaggctgtggcctttgaggagaaggaggggaaggccgtgctaaacctgctcttctccccgagggccaccaagccctcggcgctgtcccgagctgtgaaggtgtttgagacgtttgaagccaaaatccaccatctagagacccggcccgcccagaggccgcgagctgggggcccccacctggagtacttcgtgcgcctcgaggtgcgccgaggggacctggccgccctgctcagtggtgtgcgccaggtgtcagaggacgtgcgcagccccgcggggcccaaggtcccctggttcccaagaaaagtgtcagagctggacaagtgtcatcacctggtcaccaagttcgaccctgacctggacttggaccacccgggcttctcggaccaggtgtaccgccagcgcaggaagctgattgctgagatcgccttccagtacaggcacggcgacccgattccccgtgtggagtacaccgccgaggagattgccacctggaaggaggtctacaccacgctgaagggcctctacgccacgcacgcctgcggggagcacctggaggcctttgctttgctggagcgcttcagcggctaccgggaagacaatatcccccagctggaggacgtctcccgcttcctgaaggagcgcacgggcttccagctgcggcctgtggccggcctgctgtccgcccgggacttcctggccagcctggccttccgcgtgttccagtgcacccagtatatccgccacgcgtcctcgcccatgcactcccctgagccggactgctgccacgagctgctggggcacgtgcccatgctggccgaccgcaccttcgcgcagttctcgcaggacattggcctggcgtccctgggggcctcggatgaggaaattgagaagctgtccacgctgtactggttcacggtggagttcgggctgtgtaagcagaacggggaggtgaaggcctatggtgccgggctgctgtcctcctacggggagctcctgcactgcctgtctgaggagcctgagattcgggccttcgaccctgaggctgcggccgtgcagccctaccaagaccagacgtaccagtcagtctacttcgtgtctgagagcttcagtgacgccaaggacaagctcaggagctatgcctcacgcatccagcgccccttctccgtgaagttcgacccgtacacgctggccatcgacgtgctggacagcccccaggccgtgcggcgctccctggagggtgtccaggatgagctggacacccttgcccatgcgctgagtgccattggctaggtgcacggcgtccctgagggcccttcccaacctcccctggtcctgcactgtcccggagctcaggccctggtgaggggctgggtcccgggtgccccccatgccctccctgctgccaggctcccactgcccctgcacctgcttctcagcgcaacagctgtgtgtgcccgtggtgaggttgtgctgcctgtggtgaggtcctgtcctggctcccagggtcctgggggctgctgcactgccctccgcccttccctgacactgtctgctgccccaatcaccgtcacaataaaagaaactgtggtctcta

SEQ ID NO: 8 - DNA, 1435 bp, human
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 9 - DNA, 1526 bp, human
ccggaagtgacgcgaggctctgcggagaccaggagtcagactgtaggacgacctcgggtcccacgtgtccccggtactcgccggccggagcccccggcttcccggggccgggggaccttagcggcacccacacacagcctactttccaagcggagccatgtctggtaacggcaatgcggctgcaacggcggaagaaaacagcccaaagatgagagtgattcgcgtgggtacccgcaagagccagcttgctcgcatacagacggacagtgtggtggcaacattgaaagcctcgtaccctggcctgcagtttgaaatcattgctatgtccaccacaggggacaagattcttgatactgcactctctaagattggagagaaaagcctgtttaccaaggagcttgaacatgccctggagaagaatgaagtggacctggttgttcactccttgaaggacctgcccactgtgcttcctcctggcttcaccatcggagccatctgcaagcgggaaaaccctcatgatgctgttgtctttcacccaaaatttgttgggaagaccctagaaaccctgccagagaagagtgtggtgggaaccagctccctgcgaagagcagcccagctgcagagaaagttcccgcatctggagttcaggagtattcggggaaacctcaacacccggcttcggaagctggacgagcagcaggagttcagtgccatcatcctggcaacagctggcctgcagcgcatgggctggcacaaccgggtggggcagatcctgcaccctgaggaatgcatgtatgctgtgggccagggggccttgggcgtggaagtgcgagccaaggaccaggacatcttggatctggtgggtgtgctgcacgatcccgagactctgcttcgctgcatcgctgaaagggccttcctgaggcacctggaaggaggctgcagtgtgccagtagccgtgcatacagctatgaaggatgggcaactgtacctgactggaggagtctggagtctagacggctcagatagcatacaagagaccatgcaggctaccatccatgtccctgcccagcatgaagatggccctgaggatgacccacagttggtaggcatcactgctcgtaacattccacgagggccccagttggctgcccagaacttgggcatcagcctggccaacttgttgctgagcaaaggagccaaaaacatcctggatgttgcacggcagcttaacgatgcccattaactggtttgtggggcacagatgcctgggttgctgctgtccagtgcctacatcccgggcctcagtgccccattctcactgctatctggggagtgattaccccgggagactgaactgcagggttcaagccttccagggatttgcctcaccttggggccttgatgactgccttgcctcctcagtatgtgggggcttcatctctttagagaagtccaagcaacagcctttgaatgtaaccaatcctactaataaaccagttctgaaggtgtaaaaaaaaaaaaaaaaa

SEQ ID NO: 10 - DNA, 2321 bp, human
gtcctcaaccaagatggcgcggatggcttcaggcgcatcacgacaccggcgcgtcacgcgacccgccctacgggcacctcccgcgcttttcttagcgccgcagacggtggccgagcgggggaccgggaagcatggcccgggggtcggcggttgcctgggcggcgctcgggccgttgttgtggggctgcgcgctggggctgcagggcgggatgctgtacccccaggagagcccgtcgcgggagtgcaaggagctggacggcctctggagcttccgcgccgacttctctgacaaccgacgccggggcttcgaggagcagtggtaccggcggccgctgtgggagtcaggccccaccgtggacatgccagttccctccagcttcaatgacatcagccaggactggcgtctgcggcattttgtcggctgggtgtggtacgaacgggaggtgatcctgccggagcgatggacccaggacctgcgcacaagagtggtgctgaggattggcagtgcccattcctatgccatcgtgtgggtgaatggggtcgacacgctagagcatgaggggggctacctccccttcgaggccgacatcagcaacctggtccaggtggggcccctgccctcccggctccgaatcactatcgccatcaacaacacactcacccccaccaccctgccaccagggaccatccaatacctgactgacacctccaagtatcccaagggttactttgtccagaacacatattttgactttttcaactacgctggactgcagcggtctgtacttctgtacacgacacccaccacctacatcgatgacatcaccgtcaccaccagcgtggagcaagacagtgggctggtgaattaccagatctctgtcaagggcagtaacctgttcaagttggaagtgcgtcttttggatgcagaaaacaaagtcgtggcgaatgggactgggacccagggccaacttaaggtgccaggtgtcagcctctggtggccgtacctgatgcacgaacgccctgcctatctgtattcattggaggtgcagctgactgcacagacgtcactggggcctgtgtctgacttctacacactccctgtggggatccgcactgtggctgtcaccaagagccagttcctcatcaatgggaaacctttctatttccacggtgtcaacaagcatgaggatgcggacatccgagggaagggcttcgactggccgctgctggtgaaggacttcaacctgcttcgctggcttggtgccaacgctttccgtaccagccactacccctatgcagaggaagtgatgcagatgtgtgaccgctatgggattgtggtcatcgatgagtgtcccggcgtgggcctggcgctgccgcagttcttcaacaacgtttctctgcatcaccacatgcaggtgatggaagaagtggtgcgtagggacaagaaccaccccgcggtcgtgatgtggtctgtggccaacgagcctgcgtcccacctagaatctgctggctactacttgaagatggtgatcgctcacaccaaatccttggacccctcccggcctgtgacctttgtgagcaactctaactatgcagcagacaagggggctccgtatgtggatgtgatctgtttgaacagctactactcttggtatcacgactacgggcacctggagttgattcagctgcagctggccacccagtttgagaactggtataagaagtatcagaagcccattattcagagcgagtatggagcagaaacgattgcagggtttcaccaggatccacctctgatgttcactgaagagtaccagaaaagtctgctagagcagtaccatctgggtctggatcaaaaacgcagaaaatacgtggttggagagctcatttggaattttgccgatttcatgactgaacagtcaccgacgagagtgctggggaataaaaaggggatcttcactcggcagagacaaccaaaaagtgcagcgttccttttgcgagagagatactggaagattgccaatgaaaccaggtatccccactcagtagccaagtcacaatgtttggaaaacagcctgtttacttgagcaagactgataccacctgcgtgtcccttcctccccgagtcagggcgacttccacagcagcagaacaagtgcctcctggactgttcacggcagaccagaacgtttctggcctgggttttgtggtcatctattctagcagggaacactaaaggtggaaataaaagattttctattatggaaataaagagttggcatgaaagtggctactgaaaaaaaaaaaaaaaaaaaaaaaaa

SEQ ID NO: 11 - DNA, 1921 bp, human
ggcggaagtgacattatcaacgcgcgccaggggttcagtgaggtcgggcaggttcgctgtggcgggcgcctgggccgccggctgtttaacttcgcttccgctggcccatagtgatctttgcagtgacccagcatcactgtttcttggcgtgtgaagataacccaaggaattgaggaagttgctgagaagagtgtgctggagatgctctaggaaaaaattgaatagtgagacgagttccagcgcaagggtttctggtttgccaagaagaaagtgaacatcatggatcagaacaacagcctgccaccttacgctcagggcttggcctcccctcagggtgccatgactcccggaatccctatctttagtccaatgatgccttatggcactggactgaccccacagcctattcagaacaccaatagtctgtctattttggaagagcaacaaaggcagcagcagcaacaacaacagcagcagcagcagcagcagcagcaacagcaacagcagcagcagcagcagcagcagcagcagcagcagcagcagcagcagcagcagcagcaacaggcagtggcagctgcagccgttcagcagtcaacgtcccagcaggcaacacagggaacctcaggccaggcaccacagctcttccactcacagactctcacaactgcacccttgccgggcaccactccactgtatccctcccccatgactcccatgacccccatcactcctgccacgccagcttcggagagttctgggattgtaccgcagctgcaaaatattgtatccacagtgaatcttggttgtaaacttgacctaaagaccattgcacttcgtgcccgaaacgccgaatataatcccaagcggtttgctgcggtaatcatgaggataagagagccacgaaccacggcactgattttcagttctgggaaaatggtgtgcacaggagccaagagtgaagaacagtccagactggcagcaagaaaatatgctagagttgtacagaagttgggttttccagctaagttcttggacttcaagattcagaatatggtggggagctgtgatgtgaagtttcctataaggttagaaggccttgtgctcacccaccaacaatttagtagttatgagccagagttatttcctggtttaatctacagaatgatcaaacccagaattgttctccttatttttgtttctggaaaagttgtattaacaggtgctaaagtcagagcagaaatttatgaagcatttgaaaacatctaccctattctaaagggattcaggaagacgacgtaatggctctcatgtacccttgcctcccccacccccttctttttttttttttaaacaaatcagtttgttttggtacctttaaatggtggtgttgtgagaagatggatgttgagttgcagggtgtggcaccaggtgatgcccttctgtaagtgcccaccgcgggatgccgggaaggggcattatttgtgcactgagaacaccgcgcagcgtgactgtgagttgctcataccgtgctgctatctgggcagcgctgcccatttatttatatgtagattttaaacactgctgttgacaagttggtttgagggagaaaactttaagtgttaaagccacctctataattgattggactttttaattttaatgtttttccccatgaaccacagtttttatatttctaccagaaaagtaaaaatcttttttaaaagtgttgtttttctaatttataactcctaggggttatttctgtgccagacacattccacctctccagtattgcaggacagaatatatgtgttaatgaaaatgaatggctgtacatatttttttctttcttcagagtactctgtacaataaatgcagtttataaaagtgttagattgttgttaaaaaaaaaaaaaaaaaa

SEQ ID NO: 12 - DNA, 987 bp, human
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 13 - DNA, 20 bp, Artificial Sequence
  primer
ccaatccctttatgctgacg

SEQ ID NO: 14 - DNA, 26 bp, Artificial Sequence
  primer
ggaacgattaagttctctcctatttg

SEQ ID NO: 15 - DNA, 20 bp, Artificial Sequence
  primer
tggggacactgcctattttg

SEQ ID NO: 16 - DNA, 18 bp, Artificial Sequence
  primer
ttctggggtcctctgcac

SEQ ID NO: 17 - DNA, 19 bp, Artificial Sequence
  primer
ctggagaagggggaggagt

SEQ ID NO: 18 - DNA, 19 bp, Artificial Sequence
  primer
gccgatggatcactttggt

SEQ ID NO: 19 - DNA, 18 bp, Artificial Sequence
  primer
gaggatgctgctgccaag

SEQ ID NO: 20 - DNA, 21 bp, Artificial Sequence
  primer
ggcactttccttaggtttggt

SEQ ID NO: 21 - DNA, 20 bp, Artificial Sequence
  primer
aaggacaagaagcgaagcat

SEQ ID NO: 22 - DNA, 20 bp, Artificial Sequence
  primer
ttcctgtcatcccctggata

SEQ ID NO: 23 - DNA, 21 bp, Artificial Sequence
  primer
ctaccccgacatctacactcg

SEQ ID NO: 24 - DNA, 19 bp, Artificial Sequence
  primer
ctcctgcttgcgaaacttg

SEQ ID NO: 25 - DNA, 18 bp, Artificial Sequence
  primer
tcagtgacgccaaggaca

SEQ ID NO: 26 - DNA, 19 bp, Artificial Sequence
  primer
gtacgggtcgaacttcacg

SEQ ID NO: 27 - DNA, 24 bp, Artificial Sequence
  primer
tgaccttgatttattttgcatacc

SEQ ID NO: 28 - DNA, 20 bp, Artificial Sequence
  primer
cgagcaagacgttcagtcct

SEQ ID NO: 29 - DNA, 19 bp, Artificial Sequence
  primer
ctgaaagggccttcctgag

SEQ ID NO: 30 - DNA, 22 bp, Artificial Sequence
  primer
cagactcctccagtcaggtaca

SEQ ID NO: 31 - DNA, 20 bp, Artificial Sequence
  primer
cgccctgcctatctgtattc

SEQ ID NO: 32 - DNA, 20 bp, Artificial Sequence
  primer
tccccacagggagtgtgtag

SEQ ID NO: 33 - DNA, 23 bp, Artificial Sequence
  primer
gaacatcatggatcagaacaaca

SEQ ID NO: 34 - DNA, 20 bp, Artificial Sequence
  primer
atagggattccgggagtcat

SEQ ID NO: 35 - DNA, 19 bp, Artificial Sequence
  primer
ttctggcctggaggctatc

SEQ ID NO: 36 - DNA, 23 bp, Artificial Sequence
  primer
tcaggaaatttgactttccattc